(19) United States
(12) Patent Application Publication
(10) Pub. No.: US 2025/0002980 A1
(43) Pub. Date: Jan. 2, 2025

(54) QUANTITATIVE AND AUTOMATED PERMEABILIZATION PERFORMANCE EVALUATION FOR SPATIAL TRANSCRIPTOMICS

(71) Applicant: 10x Genomics, Inc., Pleasanton, CA (US)

(72) Inventors: Augusto Manuel Tentori, Dublin, CA (US); Rajiv Bharadwaj, Pleasanton, CA (US); Hanyoup Kim, Foster City, CA (US); Siyuan Xing, Newark, CA (US)

(21) Appl. No.: 18/884,272

(22) Filed: Sep. 13, 2024

Related U.S. Application Data
(63) Continuation of application No. 17/796,463, filed on Jul. 29, 2022, now Pat. No. 12,129,516.
(60) Provisional application No. 62/989,062, filed on Mar. 13, 2020.
(60) Provisional application No. 62/971,711, filed on Feb. 7, 2020.

(51) Int. Cl. C12Q 1/6837; G02B 21/36

(52) U.S. Cl. CPC C12Q 1/6837; G02B 21/367; G06T 2207/10056

(57) ABSTRACT

Quantitative methods for optimizing the permeabilization of cellular tissues for spatial transcriptomics are provided. Also provided is an instrument for quantitatively optimiz-ing the permeabilization of cellular tissues used for spatial transcriptomics.

### CROSS-REFERENCE TO RELATED APPLICATIONS

This application is a continuation of U.S. application Ser. No. 17/796,463, filed on Jul. 29, 2022, which is a U.S. National Phase filing under 35 U.S.C. § 371 of International Application No. PCT/US2021/016833, filed Feb. 5, 2021, and published as WO 2021/158925 Al on Aug. 12, 2021, which claims the benefit of priority to U.S. Provisional Application No. 62/971,711, filed Feb. 7, 2020, and to United States Provisional Application No. 62/989,062, filed Mar. 13, 2020, each of which is incorporated herein by reference in their entirety.

### FIELD OF THE INVENTION

This invention relates to an improved method of quantifying, evaluating and optimizing permeabilization techniques used in spatial transcriptomics.

### BACKGROUND OF THE INVENTION

Messenger RNA (“mRNA”) is a family of RNA molecules that convey genetic information from DNA to the ribosomes, where they specify the amino acid sequence of protein products of gene expression. Spatial transcriptomics is a technology used to spatially resolve RNA-sequence data, including all mRNA's, present in individual tissue sections. Spatially barcoded reverse transcription primers are applied in ordered fashion to the surfaces of microscope slides referred to as gene expression assay slides, thus enabling the encoding and maintenance of positional information throughout the mRNA sample processing and sequencing. When a fresh-frozen tissue section is attached to the gene expression slide, the spatially barcoded primers bind and capture mRNAs from the adjacent tissue. Post mRNA capture, reverse transcription of the mRNA occurs, and the resulting cDNA library incorporates the spatial barcode and preserves spatial information. The barcoded cDNA library enables data for each mRNA transcript to be mapped back to its point of origin in the tissue section.

Permeabilization is a process of making a membrane or cell wall permeable. Permeabilization conditions can greatly affect the ability of the GEX assay slide to capture mRNAs. Permeabilization can be accomplished using surfactants or reagents that reduce the surface tension of the membrane or cell wall and/or dissolve lipids from the cell membranes, making them permeable to antibodies. Reagents include various buffers, salts, enzymes and detergents. Permeabilization can also be accomplished by selective puncture of the cell wall or membrane. When reagents are used, permeabilization is also a function of time and temperature. Higher temperatures and longer exposure times to the reagents increase the amount of permeabilization.

In order to provide access to and detect intracellular antigens, cells must first be permeabilized using a selection of reagent, time, temperature and other conditions. Typically, the conditions affecting permeabilization tend to be optimized for each type of cellular tissue, for example using a tissue optimization (TO) assay slide. The conventional tissue permeabilization assay technique enables choosing optimal conditions based on a qualitative assessment of the appearance of a fluorescent cDNA signal that results from the assay. Samples of fresh frozen tissue are placed on TO slides having six, eight, or another number of square regions that have been coated with oligonucleotide capture probes. A single tissue section is placed on each region. The tissue can optionally be treated with a fixing agent which stabilizes the tissue for the permeabilization process, optionally stained with a suitable coloring agent that amplifies the visible features of the tissue and imaged using photomicrographs. Permeabilization reagents are added to permeabilize the tissue and enable RNA from the tissue sections to hybridize to adjacent RNA-capture probes.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

The following U.S. patents and U.S. published patent applications are each incorporated by reference in their entirety into this application:

U.S. Pat. No. 9,593,365 (Ser. No. 14/434,274), issued Mar. 14, 2017 and titled, “Methods and Product for Optimising Localized or Spatial Detection of Gene expression in a Tissue Sample”; U.S. Pat. No. 10,030,261 (Ser. No. 14/111,482), issued Jul. 24, 2018 and titled, “Method and Product for Localized or Spatial Detection of Nucleic Acid in a Tissue Sample”; and U.S. Pat. No. 10,774,374 (Ser. No. 15/565,637), published Jul. 4, 2019 and titled, “Spatially Distinguished, Multiplex Nucleic Acid Analysis of Biological Specimens.

Other references listed throughout the application are incorporated by reference in their entirety.

### SUMMARY OF THE INVENTION

Disclosed here are methods for optimizing permeabilization of cellular tissues for spatial transcriptomics. In some examples, the methods use a series of different permeabilization conditions (time, temperature, reagent type and concentration, etc.) for a tissue of interest. Following permeabilization, cDNA synthesis incorporating a fluorescently labelled nucleotide is performed on the tissue samples. In order to detect the cDNA that is generated, the permeabilized tissue is removed and the remaining cDNA footprint is imaged. The tissue removal is another point of optimization. Optimal permeabilization conditions result in a fluorescent cDNA footprint on the TO slide that closely resembles and preferably mirrors the tissue morphology image when the tissue is previously stained and imaged. A combination of qualitative brightness and sharpness of the fluorescence signal are used to determine the optimal conditions. Brightness is used as a proxy for the amount of transcripts captured and indicates assay sensitivity. Sharpness is used as a proxy for the resolution of the assay.

The present invention is directed to methods of optimizing the permeabilization of cellular tissues used for spatial transcriptomics. In some embodiments, the method includes the steps of:
a) selecting at least one parameter that affects permeabilization of the tissue sample;
b) preparing a plurality of sections of the tissue sample for permeabilization;
c) permeabilizing the sections of the tissue sample, wherein the permeabilization parameter is varied for the different tissue sections during permeabilization;
d) generating a cDNA footprint of each of the permeabilized tissue sections;
e) imaging the cDNA footprints;
f) determining a level of permeabilization for each tissue section by quantifying at least one image property that is determinative of the level of permeabilization; and
g) correlating the at least one image property with the variations of the permeabilization parameter to determine a value for the parameter that indicates an optimum level of the permeabilization parameter, thereby optimizing the permeabilization conditions for the tissue sample.

In some embodiments, the plurality of tissue sections comprises placing each tissue section on a separate area on a slide, wherein the slide comprises a plurality of array areas for placing tissue sections, and each array area comprising a plurality of capture probes immobilized thereon. In some embodiments, the plurality of capture probes comprise capture domains and after permeabilization analytes are released from the tissue sections and a plurality of the released analytes hybridize to the capture domains.

In some embodiments, the cDNA footprint can be generated by extending the capture domains of the capture probes using the hybridized analytes as a template to create cDNA molecules of the hybridized analytes. The capture domains can be extended enzymatically in the presence of one or more fluorescently labelled nucleotides, dATP, dCTP, dGTP or dTTP. In some embodiments, the cDNA footprint can be generated by hybridizing a primer that is complementary to the analytes hybridized to the capture domains and extending the primer enzymatically using the analytes as a template to create cDNA molecules of the hybridized analytes.

In some embodiments, the analyte can be DNA or RNA. In some embodiments, the analyte can be mRNA. In some embodiments, the capture domains comprise a poly(T) sequence, a poly(T) random sequence, a random nucleic acid sequence, a semi-random nucleic acid sequence or a non-random nucleotide sequence.

In some embodiments, the at least one parameter selected can be permeabilization time, permeabilization temperature, type of permeabilization reagent, a fixing agent used to fix the cellular tissue before permeabilization, and a staining agent used to stain the cellular tissue before permeabilization. The type of permeabilization reagent can be varied between one or more of organic solvents, cross-linking agents, detergents, enzymes, lysis reagents, and combinations thereof.

In some embodiments, the at least one image property can be image brightness, image sharpness, or both.

In some embodiments, the invention is directed to a quantitative method of optimizing the permeabilization of tissue samples used for spatial transcriptomics, comprising the steps of:
a) selecting two or more parameters that influence permeabilization in the tissue sample;
b) preparing a first plurality of sections of the tissue sample for permeabilization;
c) permeabilizing the first plurality of sections of the tissue sample, wherein the first of the two or more permeabilization parameters is varied for the different tissue sections,
d) generating a cDNA footprint of each of the first plurality of permeabilized tissue sections;
e) imaging the cDNA footprint for each of the tissue sections;
f) determining a level of permeabilization for each tissue section of the first plurality of tissue sections by quantifying at least one image property that is determinative of permeabilization;
g) correlating the at least one image property with the variations in the first parameter to determine a first value for the first parameter that yields an optimum level of permeabilization based on the first parameter;
h) preparing a second plurality of tissue sections of tissue sample for permeabilization;
i) permeabilizing the second plurality of tissue sections, wherein the second of the two permeabilization parameters is varied for the different tissue sections;
j) generating a cDNA footprint of each of the second plurality of permeabilized tissue sections;
k) imaging the cDNA footprint for each of the tissue sections;
l) determining a level of permeabilization for each tissue section of the second plurality of tissue sections by quantifying at least one image property that is determinative of permeabilization; and
m) correlating the at least one image property with the variations in the second parameter to determine a second value for the second parameter that yields an optimum level of permeabilization based on the second parameter, and based on the first and second values determine the optimal permeabilization of the tissue sample.

In some embodiments, additional tissue sections may be prepared to test additional parameters for levels of permeabilization, quantifying a relevant image property, correlating the image property with variations in the additional parameter and implementing a value for the additional parameter, along with values determined for other parameters, to optimize permeabilization of the tissue sample.

The invention may also include an instrument for quantitatively optimizing the permeabilization of tissue samples used for spatial transcriptomics, comprising:
a) an apparatus for receiving and storing data for at least one parameter that influences permeabilization in the tissue sample;
b) an apparatus for correlating variations in the at least one parameter with at least one image property that is determinative of permeabilization in the tissue sample;
c) an apparatus for determining an optimum value for the at least one parameter based on the correlation between the variations in the parameter and the image property;
d) an apparatus for storing the optimum value of the at least one parameter; and
e) an apparatus for informing a user of the optimum value of the at least one parameter.

The foregoing and other features and advantages of the invention will become further apparent from the following detailed description of the invention, read in conjunction with the Figures.

### BRIEF DESCRIPTION OF THE FIGURES

FIG. 1 schematically illustrates a template for a tissue optimization slide having eight square subsections or regions (top to bottom, left to right, numbered as 1-4 and 5-8, respectively), as used for the permeabilized tissue sample represented in FIGS. 2a-2e.

FIGS. 2a-e show photomicrographic images of a tissue optimization slide for cDNA footprints of different identified mouse tissues as a function of permeabilization time, varied in increments from top to bottom, left to right. The region of each slide outlined by a solid line box (and indicated by an arrow) was considered an optimal permeabilization result.

FIGS. 3a-3c show photomicrographic images of cDNA footprints from a permeabilized mouse brain tissue (FIG. 3a), masked permeabilized brain tissue (FIG. 3b), and a product of images 3a and 3b showing an image with the tissue pixel values intact (set at 1) and without the background pixel values (set at zero).

FIGS. 3d-3f are histograms of pixel counts versus pixel intensity for the images shown in FIGS. 3a-3c, respectively.

FIGS. 4a-4c show photomicrographic images of a tissue optimization slide representing cDNA footprints of mouse brain tissue as a function of permeabilization time after 5 minutes, 15 minutes and 30 minutes, respectively.

FIG. 5 shows a plot of net average image intensity (arbitrary fluorescent units) versus permeabilization time (minutes) for each of the images shown in FIGS. 4a-4c.

FIG. 6 shows a radial profile plot of net average image intensity (arbitrary fluorescent units) versus spatial frequency (1/pixels) for each of the images shown in FIGS. 4a-4c.

FIG. 7 shows a plot of an image sharpness metric (average spatial frequency over a selected range) versus permeabilization time for each of the images shown in FIGS. 4a-4c.

FIG. 8 shows photomicrographic images of a tissue optimization slide representing cDNA footprints of human kidney tissue as a function of permeabilization time, varied in increments from top to bottom, left to right. Region 2 was considered to have optimal brightness and sharpness. Region 4 was considered to be qualitatively optimal.

FIG. 9 is a bar graph showing pixel intensity distribution (measured in arbitrary fluorescent units, y axis) for seven statistical parameters for the eight images (x axis) from the tissue optimization slide represented in FIG. 8.

FIG. 10 is a table containing normalized metrics for the eight images (columns 1-8) from the tissue optimization slide represented in FIG. 8. The metrics normalize image sharpness, on a scale of 0 to 1 for 28 different image sharpness algorithms (rows AMCO through WAVRA).

FIG. 11 shows photomicrographic images of a tissue optimization slide representing cDNA footprints of human small intestine tissue as a function of permeabilization time, varied in increments from top to bottom, left to right. Region 3 was considered to have optimal sharpness. Regions 6-8 were considered to have optimal brightness. Regions 6 and 7 were considered to be qualitatively optimal.

FIG. 12 is a bar graph showing pixel intensity distribution (measured in arbitrary fluorescent units, y axis) for seven statistical parameters of the eight images (x axis) from the tissue optimization slide represented in FIG. 11.

FIG. 13 is a table containing normalized metrics for the eight images (columns 1-8) from the tissue optimization slide represented in FIG. 11. The metrics normalize image sharpness, on a scale of 0 to 1, for 28 different image sharpness algorithms (rows AMCO through WAVRA).

FIGS. 14 and 15 show magnifications of the sharpest image from the tissue optimization slide represented in FIG. 11, taken from Region 3 (6 minutes permeabilization time).

FIGS. 16 and 17 show magnifications of the brightest image from the tissue optimization slide represented in FIG. 11, taken from Region 6 (24 minutes permeabilization time).

FIG. 18 shows photomicrographic images of a tissue optimization slide representing cDNA footprints of mouse lung tissue as a function of permeabilization time, varied in increments from top to bottom, left to right. Region 2 was considered to have optimal sharpness. Regions 5-8 were considered to have optimal brightness and to be qualitatively optimal.

FIG. 19 is a bar graph showing pixel intensity distribution (measured in arbitrary fluorescent units, y axis) of seven statistical parameters for the eight images (x axis) from the tissue optimization slide represented in FIG. 18.

FIG. 20 is a table containing normalized metrics for the eight images (columns 1-8) from the tissue optimization slide represented in FIG. 18. The metrics normalize image sharpness, on a scale of 0 to 1, for 28 different image sharpness algorithms (rows AMCO through WAVRA).

FIG. 21 shows a magnification of the sharpest image from the tissue optimization slide represented in FIG. 18, taken from Region 2 (3 minutes permeabilization time).

FIG. 22 shows a magnification of one of the brightest images from the tissue optimization slide represented in FIG. 18, taken from Region 5 (18 minutes permeabilization time).

FIG. 23 shows photomicrographic images of a tissue optimization slide representing cDNA footprints of mouse brain tissue as a function of permeabilization time, varied in increments from top to bottom, left to right. Regions 2 and 6 were considered to have optimal sharpness and to be qualitatively optimal. Region 5 was considered to have optimal brightness.

FIG. 24 is a bar graph showing pixel intensity distribution (measured in arbitrary fluorescent units, y axis) for seven statistical parameters of the eight images (x axis) from the tissue optimization slide represented in FIG. 23.

FIG. 25 is a table containing normalized metrics for the eight images (columns 1-8) from the tissue optimization slide represented in FIG. 23. The metrics normalize image sharpness, on a scale of 0 to 1, for 28 different image sharpness algorithms (rows AMCO through WAVRA).

FIG. 26 shows photomicrographic images of a tissue optimization slide representing cDNA footprints of human liver tissue as a function of permeabilization time, varied in increments from top to bottom, left to right. Region 2 was considered to be qualitatively optimal. Region 5 was considered to have optimal brightness and sharpness.

FIG. 27 is a bar graph showing pixel intensity distribution (measured in arbitrary fluorescent units, x axis) for seven statistical parameters of the eight images (y axis) from the tissue optimization slide represented in FIG. 26.

FIG. 28 is a histogram of pixel intensity (x axis) versus pixel count (y axis) for the brightest image from the tissue optimization slide of FIG. 26, taken from Region 5 (18 minutes permeabilization time).

FIG. 29 is a table containing normalized metrics for the eight images (columns 1-8) from the tissue optimization slide represented in FIG. 26. The metrics normalize image sharpness, on a scale of 0 to 1, for 28 different image sharpness algorithms (rows AMCO through WAVRA).

FIGS. 30 and 31 represent the transformation of a photomicrographic image (FIG. 30) of a cDNA footprint on a tissue optimization slide into a spot image (FIG. 31), according to one embodiment of the invention, using original and spot cDNA footprints of permeabilized mouse lung tissue.

FIG. 32 schematically represents an exemplary array of spots taken from a section of a spot image.

FIG. 33 schematically represents an exemplary first technique for quantifying an array of image spots.

FIG. 34 schematically represents an exemplary second technique for quantifying an array of image spots.

FIGS. 35a and 35b schematically represent another example of the first technique for quantifying an array of image spots, derived from original and spot images of cDNA footprints of permeabilized mouse lung tissue, examples seen in FIG. 30 and FIG. 31.

FIGS. 36a and 36b schematically represent another example of the second technique for quantifying an array of image spots, derived from original and spot images of cDNA footprints of permeabilized mouse lung tissue, examples seen in FIG. 30 and FIG. 31.

### DETAILED DESCRIPTION OF THE INVENTION

Disclosed are methods for optimizing permeabilization of cellular tissues used for spatial transcriptomics. Various parameters that influence the permeabilization may be selected, used to permeabilize cells and to release analytes (e.g., RNAs) from the cells onto, for example, an array surface. The analytes on the array, or amplification products (e.g., cDNA) made from mRNA analytes on the array, may be imaged. Various image properties may be analyzed to determine values for the parameter tested that yield optimal permeabilization.

By correlating the image property with the variations in the parameters examined, the permeabilization can be optimized for a parameter. The method can be used to optimize the value of one parameter, to independently optimize the value of two or more parameters, or to optimize the values of two or more parameters simultaneously and in combination.

Exemplary image properties that can be quantified to determine an optimal level of permeabilization include without limitation image brightness and sharpness. Exemplary parameters that influence these image properties include without limitation permeabilization time, permeabilization temperature, composition and concentration of permeabilization reagent, tissue fixing agent, and tissue staining agent.

In one embodiment, the property to be quantified and optimized is image brightness. Image brightness is representative of the amount of transcripts captured and indicates assay sensitivity. In one embodiment, the tissue sample that has been optionally fixed and optionally stained is permeabilized on a tissue optimization slide that includes a reverse transcription primer, using selected permeabilization conditions. The reverse transcription primer can include an oligonucleotide (e.g., fluorescent DNA or RNA capture probes) as described herein. Following permeabilization, reverse transcription of the mRNA released from the permeabilized tissue sample and captured on the oligonucleotides on the array surface can be performed using procedures described in the “User Guide for Visium Spatial Gene Expression Reagent Kits—Tissue Optimization,” Rev. A, November 2019, published by 10X Genomics. The reverse transcription yields a cDNA footprint on the slide surface which can then be imaged following removal of the tissue sample from the slide. The brightness of the resulting image (which is suitably a fluorescence image) can be quantified using a variety of techniques as described herein.

In one embodiment, the areas of the image that correspond to the permeabilized tissue and the areas of the image that correspond to the background can be identified. In one implementation of this technique, a mask image can be prepared that minimizes or eliminates the background components of the image. In one example, the mask assigns a value of 1 for each pixel in the tissue region of the image and a value of zero for each pixel in the background region of the image (e.g., areas around the tissue region). The original image and the mask image can be merged to yield a tissue image that attributes a pixel value only to the tissue region of the image and minimizes the background region. The values of the pixels in the tissue image can be plotted to quantify the pixel count versus pixel intensity (histogram of pixel intensities) for the tissue region of the image. The same can be done for the background region of the tissue. Statistical parameters (such as mean, median, mode, standard deviation, root mean square error, different percentiles, quartiles, etc.) of the pixel intensity values of both the tissue and the background can then be calculated, reported, and plotted as quantitative metrics to compare the different permeabilization conditions. This procedure can be repeated using tissue samples permeabilized under various conditions of time, temperature, reagent composition and concentration in order to quantify the optimal image brightness as an aide for optimizing the permeabilization conditions of a tissue.

In another embodiment, the property to be quantified and optimized is image sharpness. Image sharpness is a proxy for the resolution of the assay. In one embodiment, image sharpness can be quantified using a variety of quantification instruments and techniques, including without limitation Fast Fourier Transform (“FFT”) approaches. Fourier Transform converts an image into its sine and cosine components and thereby represents the image in a frequency domain. If the input signal is an image, then the number of frequencies in the frequency domain is equal to the number of pixels in the image or spatial domain. FFT processes the images in two dimensions, x and y, to provide a radial profile of each image. The FFT thus displays the spatial frequency of each image, which is a measure of how often the sinusoidal components of the image repeat per unit of distance, to indicate the size of features present in the image. Smaller features coupled with higher spatial frequencies correlate with resolution or sharpness. A high level of such smaller features indicates higher resolution and a sharper image. The resulting measurements can be used to calculate metrics for resolution such as average frequency over a range, intensity of a frequency range or ratio of different frequencies, thereby serving as an aide for optimizing the permeabilization conditions of the tissue.

Image sharpness of the assay can also be quantified using any combination of the following algorithms that are used in autofocusing applications:

Absolute Central Moment, Brenner's function, Image contrast, Image curvature, DCT energy ratio, DCT reduced energy ratio, Gaussian derivative, Graylevel variance, Graylevel local variance, Normalized GLV, Energy of gradient, Thresholded gradient, Squared gradient, Helmli's mean method, Histogram entropy, Histogram range, Energy of Laplacian, Modified Laplacian, Variance of Laplacian, Diagonal Laplacian, Steerable filters, Spatial frequency, Tenengrad, Tenengrad variance, Vollath's correlation, Sum of Wavelet coefficients, Sum of Wavelet coefficients A, Variance of Wavelet, and Multi-level two-dimensional inverse FWT.

Various other techniques for quantifying image brightness and image sharpness can also be employed, as described herein.

### Definitions

Unless defined otherwise, all technical and scientific terms used herein have the same meaning as commonly understood by one of ordinary skill in the art to which the present invention pertains. It is to be understood that the terminology used herein is for describing embodiments only and is not intended to be limiting. For purposes of interpreting this disclosure, the following description of terms will apply and, where appropriate, a term used in the singular form will also include the plural form and vice versa.

Herein, “analyte” refers to a substance whose chemical constituents are being identified and/or measured. Generally, this application refers to analytes from and/or produced by cells, for example as found in tissue samples. Any or all molecules or substance from or produced by a cell may be referred to herein as analytes. Chemically, cellular analytes may include proteins, polypeptides, peptides, saccharides, polysaccharides, lipids, nucleic acids, and other biomolecules. In some examples, the analytes referred to in this application are RNAs, particularly mRNAs.

Herein, “array” refers to a region on a support that contains multiple demarcated regions of oligonucleotides, interspersed with intervening regions that do not contain oligonucleotides. In some examples, these regions may be referred to as “oligonucleotide arrays” or “capture areas”. The arrays herein generally have oligonucleotides that contain spatial barcodes and, thus, the arrays may be referred to as “spatial” arrays.

Herein, “brightness” or “image brightness” is an image property that may be used to determine permeabilization. Brightness is generally used as a proxy for the amounts of transcripts captured and indicates assay sensitivity. “Brightness” represents the fluorescence output per fluorophore.

Herein, “footprint” refers to analytes released from permeabilized cells that are part of a tissue sample. Analytes can be mRNA. In some examples, the released mRNA may be quantified by detecting immunofluorescent probes hybridized to the mRNA. In some examples, the released mRNA may be quantified by detecting cDNA that incorporates fluorescent nucleoside triphosphates using the mRNA as template. The fluorescent cDNA can therefore provide a cDNA footprint.

Herein, “fix,” refers to formation of covalent bonds, such as crosslinks, between biomolecules or within molecules. The process of fixing cells for example, is called “fixation.” The agent that causes fixation is generally referred to as a “fixative” or “fixing agent.” “Fixed cells” or “fixed tissues” refer to cells or tissues that have been in contact with a fixative under conditions sufficient to allow or result in formation of intra-and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. Non limiting examples of fixatives or fixing agents include methanol, paraformaldehyde, formalin, and acetone to name a few.

Herein, “hybridize” refers to a nucleotide sequence of a single-stranded nucleic acid molecule forming a complex with a nucleic acid molecule having a partially, substantially or fully complementary nucleotide sequence. Generally, the complex forms through hydrogen bonding between complementary nucleotide bases in separate nucleic acid molecules. Herein, “parameter” refers to a condition that may influence permeabilization of

a tissue sample.

Herein, “permeabilize” means to cause cells, generally within a tissue, to release analytes by disrupting the integrity of cellular membranes and/or cell walls.

Herein, “sample” or “biological sample” generally refers to a collection of cells or to a tissue. Generally, a tissue contains multiple cells, often similar cells that may perform the same or similar functions. The sample may be a cell sample. The sample may be a cell line or cell culture sample. The sample can include one or more cells, or one or more cell aggregates or clusters. The sample may be a tissue sample, such as a biopsy, core biopsy, needle aspirate, or fine needle aspirate. The sample may be a tissue from a diseased or cancerous organ, or one suspected of being diseased or cancerous. Example tissue types in animals may include connective, epithelial, brain, adipose, muscle and nervous tissue. The sample may be a fluid sample, such as a blood sample, urine sample, or saliva sample. The sample may be a skin sample. The sample may be a check swab. The sample may be a plasma or serum sample. In some examples, a sample may comprise any number of macromolecules, for example, cellular macromolecules or cellular analytes. The present disclosure is not limited to any particular type of tissue.

Herein, “section” generally refers to a thin layer or slice from a larger object. Generally, herein, sections are thin slices taken from tissue blocks, such as 10 um sections for example.

Herein, “sharpness” or “image sharpness” is an image property that may be used to determine permeabilization. Sharpness is generally used as a proxy for the resolution of the assay.

### Obtaining Spatially Aligned Analyte Expression Data from Cells and Tissues

Spatial analysis methodologies and compositions described herein can provide a vast amount of analyte and/or expression data for a variety of analytes within a biological sample at high spatial resolution. Spatial analysis methods and compositions can include, e.g., the use of a capture probe including a spatial barcode (e.g., a nucleic acid sequence that provides information as to the location or position of an analyte within a cell or a tissue sample, including a mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample, it serves as a proxy for the analyte.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348 (6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. The above references, if US Patents or US Patent Publications, are incorporated herein by reference in their entirety. Further non-limiting aspects of spatial analysis methodologies and compositions are described herein.

Array-based spatial analysis methods involve the transfer of one or more analytes from a biological sample to an array of features (e.g., spots) on a substrate, where each feature is associated with a plurality of capture probes that provide a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent, including a ligation product or an analyte capture agent, or a portion thereof), or derivatives thereof (see, e.g., Section (II)(b) (vii) of WO 2020/176788 and/or U.S. Patent Application Publication No. 2020/0277663 regarding extended capture probes; incorporated herein by reference in their entirety). In some cases, capture probes may be configured to form ligation products with a template (e.g., a DNA or RNA template, such as an analyte or an intermediate agent, or portion thereof), thereby creating ligation products that serve as proxies for a template. As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

In some cases, spatial analysis can be performed by detecting multiple oligonucleotides that hybridize to an analyte. In some instances, for example, spatial analysis can be performed using RNA-templated ligation (RTL). Methods of RTL have been described previously (See, e.g., Credle et al., Nucleic Acids Res. 2017 Aug. 21;45(14):e128). Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level.

In some cases, a map of analyte presence and/or level can be aligned to an image of a biological sample using one or more fiducial markers (e.g., objects placed in the field of view of an imaging system which appear in the image produced, as described in the Substrate Attributes Section and Control Slide for Imaging Section of WO 2020/123320). Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Systems and methodologies in the field of spatial transcriptomics are designed to obtain spatially resolved analyte expression data (e.g., genomics, proteomics, transcriptomics) from tissues. In some examples, a tissue may be overlaid onto a support comprising barcoded oligonucleotides or capture probes. Generally, the oligonucleotides comprise a spatial barcode, which is correlated with and is an identifier for the location of the particular oligonucleotide on the support (e.g., in some examples, oligonucleotides having known barcode sequences are printed onto designated areas of the support). When analytes are released from a biological sample and migrate toward and contact the barcoded oligonucleotides, the barcoded oligonucleotides capture, or hybridize to, the analytes. In some examples, mRNAs may be the analytes and barcoded oligonucleotides may capture mRNAs having specific nucleotide sequences by hybridization, for example the barcoded oligonucleotides comprise a poly(T) capture domain that can hybridize a poly(A) tail of a mRNA. In the examples where mRNA is the analyte, reverse transcription of the captured mRNA can be initiated using added primers, and cDNA is produced using the barcoded oligonucleotide as a template. The resultant cDNA that is synthesized incorporates the barcodes included in the barcoded oligonucleotide or capture probe. The cDNAs may be amplified. A library of the cDNAs/amplified cDNAs is prepared and nucleotide sequences of the libraries are obtained. Nucleotide sequences of the spatial barcodes provides for the data for an mRNA transcript to be mapped back to its location on the support, and by also obtaining an image of the tissue and cells overlaid onto the support at the beginning of the procedure, mRNA transcripts may be mapped to the location in the overlaid tissue, where the mRNA was expressed.

In some examples, a planar support on the surface of which is attached a spatially ordered arrangement of barcoded oligonucleotides comprising analyte capture domains is used. In some examples, an analyte capture domain may be an oligo(dT) sequence for capturing poly(A) sequences of eukaryotic mRNA. Other sequences may be used to capture specific nucleic acids, including specific mRNAs. The arrangement of the oligonucleotides on the surface of the support can be known because the oligonucleotides comprise spatial barcodes. In some examples, the oligonucleotides, with known spatial barcodes, are printed in a known pattern onto specific, known areas of the surface of the planar support in a predetermined arrangement. A tissue is then applied to the surface of the support and analytes (e.g., mRNA) are released from the cells that make up the tissue. mRNAs released from the tissue migrate to the surface of the support and hybridize to oligo(dT) capture domain sequences of the attached oligonucleotides. The hybridized mRNAs are amplified using reverse transcription into complementary oligonucleotides that include sequences from the captured mRNA linked to the spatial barcode of the oligonucleotide to which the mRNA bound. Obtaining and decoding the nucleotide sequences of the complementary oligonucleotides reveals where on the support specific mRNAs bound to oligonucleotides. These locations are then correlated to regions of the tissue that was applied to the surface of the support.

In modifications of the above method, a tissue sample may be probed for expression of specific proteins using antibodies. The antibodies may have attached nucleotide tags having a specific nucleotide sequence that capture domains of the barcoded molecules on a support are designed to capture through hybridization. Thus, proteomic data can be obtained from the oligonucleotide arrays.

In modifications of the above method, a tissue sample may be probed for presence or absence of genetic mutations, variants, diversity, polymorphisms and the like in genomes, including single-nucleotide polymorphisms (SNPs) or single-nucleotide variants

(SNVs) in genomes of cells making up the tissue. In some examples, a probe for a SNP or SNV may include a specific nucleotide sequence that can differentially hybridize to a genomic sequence dependent on whether a SNP or SNV is present. In some examples, a probe for a SNP or SNV may include a nucleotide sequence that can hybridize to a genomic sequence that is linked to (e.g., upstream of downstream of) a genomic region that might contain the SNP or SNV. Extension of the hybridized sequence, using the region of the genome that might contain the SNP/SNV as a template, and nucleotide sequencing of the extension product, may be used to determine if the SNP/SNV is present in the extension product. In some examples, probes for specific SNPS or SNVs may be part of the capture domain of certain oligonucleotides that make up the oligonucleotide array. Other techniques may be used to detect SNPs and/or SNVs.

In modifications of the above method, a tissue sample may be probed for isoforms of genes, transcripts (e.g., alternative transcription start sites, alternatively spliced mRNAs) or proteins. In some examples, a probe for an isoform of a gene or transcript may be designed to hybridize to one form but not the other, or may be designed to hybridize to or near a region that may contain the isoform such that amplification and/or extension of the hybridized probe, and optional nucleotide sequencing of the amplified product, can detect presence or absence of specific isoforms. In some examples, a probe for an isoform of a protein may be an antibody designed to differentially bind to the different isoforms. The antibodies used may have attached nucleotide tags that can capture domains of the barcoded molecules on a support, as described above.

### Cell Permeabilization in Spatial Transcriptomics

The ability of a spatial array to capture an analyte and determine its spatial location within a tissue sample is dependent on many factors. One of those factors is the ability of the analyte or analyte proxy to leave the tissue sample and hybridize to the capture domain of the capture probe. The present invention is directed to a quantitative method of optimizing the permeabilization of cellular tissues used for spatial transcriptomics, thereby enhancing the ability of an analyte within a tissue sample to migrate from the tissue sample and bind to the capture domain of a capture probe on the spatial array.

Common parameters that influence permeabilization include, without limitation, permeabilization time, permeabilization temperature, permeabilization reagent type and concentration, tissue preparation (including tissue fixing agent and tissue staining agent), and later removal of the tissue from the slide. Permeabilization methods can also be varied between chemical permeabilization (using reagents), mechanical permeabilization (e.g., magnetic stirring, etc.) and electrophoresis. Optimum values for the permeabilization parameters may vary depending on the tissue type. Optimizing permeabilization conditions for each tissue sample type can be very beneficial in capturing as many analytes as possible from any given tissue sample type.

Types of tissue samples for which optimized permeabilization can be beneficial using the method of the invention include without limitation mammalian brain tissue, mammalian olfactory bulb tissue, mammalian spleen tissue, mammalian lung tissue, mammalian esophageal tissue, mammalian skin tissue, mammalian liver tissue, mammalian testicular tissue, mammalian ovarian tissue, mammalian bone tissue, mammalian heart tissue, mammalian abdominal tissue, and mammalian intestinal tissue. Optimal values for permeabilization parameters vary depending on the type of mammal that produces the tissue. For example, the optimal permeabilization conditions for human lung tissue can differ from the optimal permeabilization conditions for mouse lung tissue. Further, within a species there may be differences based on the individual from which the tissued originated.

### Permeabilization Time

Optimal permeabilization time is influenced not only by the tissue sample type, but also by the values for other permeabilization parameters such as temperature, reagent type and concentration. Depending on these other parameters, the optimal permeabilization time can be between about 3 minutes and about 90 minutes, suitably between about 5 minutes and about 60 minutes. In accordance with the invention, the permeabilization time can be quantified and optimized by first varying the time in suitable increments within that range. For example, the permeabilization time can be varied in increments of about three minutes, or about four minutes, or about five minutes, or about six minutes, or about eight minutes, or about ten minutes, or about twelve minutes for different sections of a tissue sample to determine the optimal permeabilization time for a given tissue type and set of conditions.

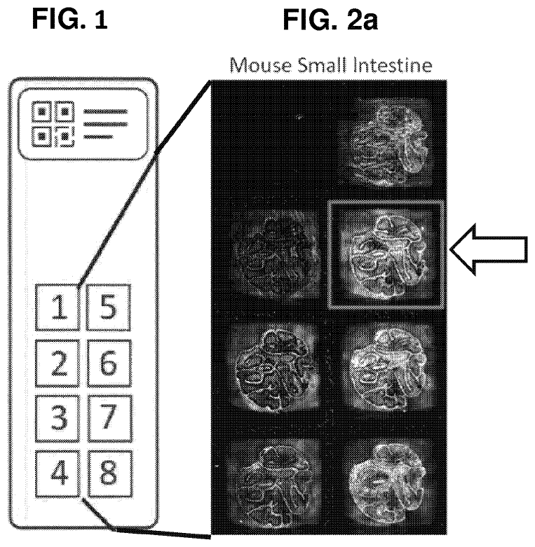

FIG. 1 illustrates an exemplary tissue optimization (TO) slide that can be used to vary the permeabilization time, temperature and other parameters for different tissue sample types. The TO slide is divided into eight rectangular capture areas, numbered 1 through 8. Each capture area is coated with a lawn of capture probes. The capture probes include capture domains comprising a single-stranded sequence of poly-deoxythymine (dT) (i.e., polyT), designed to capture poly(A) tail sequences of eukaryotic mRNAs. In some examples, capture domains may be other nucleotide sequences designed to be complementary to specific RNAs or DNAs and to capture those RNAs or DNAs through hybridization. A prepared tissue section (e.g., fresh frozen tissue section, FFPE tissue section, etc.) can be place on the array slide. The tissue section can be stained with a suitable dye as described below, and imaged. When hematoxylin and eosin (H&E) are used as the dye, the hematoxylin stains cell nuclei blue, and eosin stains the extracellular matrix and cytoplasm pink, with other structures assuming different shades, hues and combinations of these colors. A permeabilization reagent can be added to the tissue on the array.

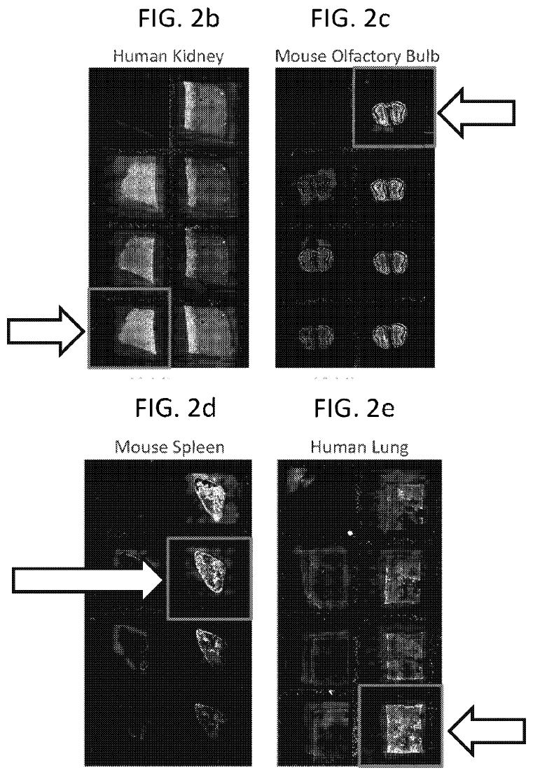

FIGS. 2a-2e demonstrate the various times that can be examined for optimization of different cellular tissue samples. For each tissue type, eight tissue sections were initially prepared using methanol as the fixing agent and no staining agent. The tissue sections were permeabilized for various times using a permeabilization temperature of 37 degrees C. and using a combination of proteinase-K with 10% by weight sodium dodecyl sulfate as the permeabilization reagent. The permeabilization times for the tissue samples placed in each of the capture regions 1-8 were as follows: Region 1—negative control (no permeabilization), Region 2—3 minutes, Region 3—6 minutes, Region 4—12 minutes, Region 5—18 minutes, Region 6—24 minutes, Region 7—30 minutes, Region 8—36 minutes. The arrow in each of the tissue type photomicrographs in FIGS. 2a-2c point to the optimal permeabilization times for each kind of tissue, represented by the best combination of image brightness and sharpness. Fluorescent cDNA synthesis was carried out, as described later in this document.

FIGS. 2a-2e represent photomicrographic images that capture the fluorescence resulting from the fluorescently labelled cDNA synthesis. The optimum times varied by a factor of three and were 24 minutes for mouse small intestine tissue (FIG. 2a), 12 minutes for human kidney tissue (FIG. 2b), 18 minutes for mouse olfactory bulb tissue (FIG. 2c), 24 minutes for mouse spleen tissue (FIGS. 2d) and 36 minutes for human lung tissue (FIG. 2e). FIGS. 2a-2e provide examples of qualitative optimization in which the permeabilization time is optimized by viewing the photomicrographs for brightness and sharpness. Optimization according to the invention further involves quantifying the image properties as described below.

### Permeabilization Temperature

Other parameters that can be optimized include permeabilization temperature, which typically ranges from about 4 degrees C. to about 50 degrees C., depending on the tissue type, permeabilization reagent, and other variables. In order to optimize the permeabilization temperature for a given tissue type and set of conditions, the permeabilization temperature can be varied within this range in increments of about 1 degree, or about 2 degrees, or about 3 degrees, or about 4 degrees, or about 5 degrees, or about 6 degrees, or about 8 degrees, or about 10 degrees, or about 12 degrees Celsius. When two or more parameters such as permeabilization time and temperature are to be optimized, the parameters can be optimized simultaneously or in sequence.

An iterative technique may be employed for simultaneous optimization of two or more parameters. Using the foregoing examples, permeabilization time may be optimized at 37 degrees C. for a given tissue type, such as mouse spleen, resulting in an optimum time of 24 minutes as shown in FIG. 2c. Then, using the 24-minute time, a next set of cellular tissue sections can be permeabilized at selected temperature increments above and below 37 degrees C. This may result in discovery of a temperature yielding somewhat better permeabilization. Using that newly discovered temperature, a next set of cellular tissue samples can be permeabilized by varying the time in small increments above and below 24 minutes. This might result in discovery of a permeabilization time at the new temperature that yields still better permeabilization. These iterations can be repeated until the best permeabilization based on both time and temperature is achieved. Similar iterations can be performed for simultaneous optimization of reagents, reagent concentrations, and other parameters.

### Permeabilization Reagent

Another parameter that can be optimized according to the method of the invention is the type of permeabilization reagent. Suitable permeabilization reagents include without limitation one or more organic solvents (e.g., acetone, ethanol, and methanol), detergents (e.g., saponin, Triton X-100™ (C₁₄—H₂₂—O—(C₂—H₄—O)ₙ), Tween-20™ (polyoxyethylene sorbitan monolaurate), sodium dodecyl sulfate (SDS), and enzymes (e.g., trypsin, collagenase, proteases such as pepsin or proteinase K). In some embodiments, the detergent is an anionic detergent (e.g., SDS or N-lauroylsarcosine sodium salt solution). In some embodiments, the tissue section can be permeabilized using any of the detergents described herein before, concurrent or after treatment with any of the enzymes described herein.

In some embodiments, the permeabilization reagent can be an aqueous solution of sodium dodecyl sulfate (SDS) and/or N-lauroylsarcosine or N-lauroylsarcosine sodium salt. The tissue section can be incubated with and permeabilized using such reagents in a concentration of greater than about 1.0 w/v %, or greater than about 2.0 w/v %, or greater than about 3.0 w/v %, or greater than about 4.0 w/v %, or greater than about 5.0 w/v %, greater than about 6.0 w/v %, or greater than about 7.0 w/v %, or greater than about 8.0 w/v %, or greater than about 9.0 w/v %, or greater than about 10.0 w/v %, or greater than about 11.0 w/v %, or greater than about 12.0 w/v %, or greater than about 13.0 w/v %. Specific concentration ranges can be about 1.0 w/v % to about 14.0 w/v %, or about 2.0 w/v % to about 14.0 w/v %, or about 2.0 w/v % to about 12.0 w/v %, or about 2.0 w/v % to about 10.0 w/v %, or about 4.0 w/v % to about 14.0 w/v %, or about 4.0 w/v % to about 12.0 w/v %, or about 4.0 w/v % to about 10.0 w/v %, or about 6.0 w/v % to about 14.0 w/v %, or about 6.0 w/v % to about 12.0 w/v %, or about 6.0 w/v % to about 10.0 w/v %, or about 8.0 w/v % to about 14.0 w/v %, or about 8.0 w/v % to about 12.0 w/v %, or about 8.0 w/v % to about 10.0 w/v %, or about 10.0% w/v % to about 14.0 w/v %, or about 10.0 w/v % to about 12.0 w/v %, or about 12.0 w/v % to about 14.0 w/v %.

In some embodiments, the permeabilization reagent can be one or more lysis reagents. Examples of suitable lysis reagents include, but are not limited to, bioactive reagents such as lysis enzymes that are used for lysis of different tissue cell types, including without limitation lysozymes, achromopeptidase, lysostaphin, labiase, kitalase, lyticase, and a variety of other commercially available lysis enzymes. Other lysis reagents can additionally or alternatively be added to the biological sample to facilitate permeabilization. For example, surfactant-based lysis solutions can be used to lyse tissue cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the cellular tissue can be permeabilized by non-chemical permeabilization methods. Non-chemical permeabilization methods include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce permeabilization of the tissue cells.

In some embodiments, a permeabilization reagent solution may contain one or more proteases. A tissue section treated with a protease capable of degrading histone proteins can result in the generation of fragmented genomic DNA. The fragmented genomic DNA can be captured using the same capture domain (e.g., capture domain having a poly(T) sequence) used to capture mRNA, for example by using a proxy for the DNA that includes a poly(A) tail. In some embodiments, a tissue can be treated with a protease capable of degrading histone proteins and an RNA protectant prior to spatial profiling in order to facilitate the capture of both genomic DNA and mRNA. As used herein, the term “histone protein” typically refers to a linker histone protein (e.g., H1) and/or a core histone protein (e.g., H2A, H2B, H3, and H4).

In some instances, a protease reagent can be used to degrade linker histone proteins and/or core histone proteins. Any suitable protease capable of degrading histone proteins in a cellular tissue can be used. Non-limiting examples of proteases capable of degrading histone proteins include proteases inhibited by leupeptin and TLCK (Tosyl-L-lysyl-chloromethane hydrochloride), a protease encoded by the EUO gene from Chlamydia trachomatis serovar A, granzyme A, a serine protease (e.g., trypsin or trypsin-like protease, neutral serine protease, elastase, cathepsin G), an aspartyl protease (e.g., cathepsin D), a peptidase family Cl enzyme (e.g., cathepsin L), pepsin, proteinase K, a protease that is inhibited by the diazomethane inhibitor Z-Phe-CHN(2) or the epoxide inhibitor E-64, a lysosomal protease, or an azurophilic enzyme (e.g., cathepsin G, elastase, proteinase 3, neutral serine protease). In some embodiments, a serine protease is a trypsin enzyme, trypsin-like enzyme or a functional variant or derivative thereof.

In some embodiments, the protease reagent can include a trypsin enzyme such as P00761, P00760, Q29463, or a combination thereof. In some embodiments, a protease reagent capable of degrading one or more histone proteins includes an amino acid sequence with at least 80% sequence identity to P00761, P00760, or Q29463. In some embodiments, a protease reagent capable of degrading one or more histone proteins includes an amino acid sequence with at least 85%, 90%, 91%, 92%, 93%, 94%, 95%, 96%, 97%, 98%, or 99% identity to P00761 (SEQ ID NO: 8), P00760 (SEQ ID NO: 9), or Q29463 (SEQ ID NO: 10). A protease reagent may be considered a functional variant if it has at least 50% e.g., at least 60%, 70%, 80%, 90%, 95%, 96%, 97%, 98%, 99%, or 100% of the activity relative to the activity of the normal or wild-type protease. In some embodiments, the protease reagent can include enzymatic treatment with pepsin enzyme, or pepsin like enzyme.

In some embodiments, the protease reagent can include a pepsin enzyme such as P00791/PEPA_PIG; P00792/PEPA_BOVIN, functional variants, derivatives, or combinations thereof. Additionally, the protease reagent may be contained in a reaction mixture (solution), which also includes other components (e.g., buffer, salt, chelator (e.g., EDTA), and/or detergent (e.g., SDS, N-lauroylsarcosine sodium salt solution)). The reaction mixture may be buffered, having a pH of about 6.5-8.5, e.g., about 7.0-8.0.

The permeabilization reagent solution can contain additional reagents or the tissue section may be treated with additional reagents in order to optimize biological sample permeabilization. In some embodiments, an additional permeabilization reagent is an RNA protectant. As used herein, the term “RNA protectant” refers to a reagent that protects RNA from RNA degradation by, for example, nucleases (e.g., RNases). Any appropriate RNA protectant that protects RNA from degradation can be used. A non-limiting example of a RNA protectant includes organic solvents (e.g., at least 60%, 65%, 70%, 75%, 80%, 85%, 90%, or 95% v/v organic solvent), which include, without limitation, ethanol, methanol, propan-2-ol, acetone, trichloroacetic acid, propanol, polyethylene glycol, acetic acid, or a combination thereof. In some embodiments, an RNA protectant includes ethanol, methanol and/or propan-2-ol, or a combination thereof. In some embodiments, an RNA protectant includes RNA-later ICE (ThermoFisher Scientific). In some embodiments, the RNA protectant includes at least about 60% ethanol. In some embodiments, the RNA protectant includes about 60-95% ethanol, about 0-35% methanol and about 0-35% propan-2-ol, wherein the total amount of organic solvent in the medium is not more than about 95%. In some embodiments, the RNA protectant includes about 60-95% ethanol, about 5-20% methanol and about 5-20% propan-2-ol, wherein the total amount of organic solvent in the medium is not more than about 95%.

In some embodiments, the RNA protectant may be combined with a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the salt is a sulfate salt, for example, ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, or zinc sulfate. In some embodiments, the salt is ammonium sulfate. The salt may be present at a concentration of about 20 g/100 ml of medium or less, such as about 15 g/100 ml, 10 g/100 ml, 9 g/100 ml, 8 g/100 ml, 7 g/100 ml, 6 g/100 ml, 5 g/100 ml or less, e.g., about 4 g, 3 g, 2 g or 1 g/100 ml.

Additionally, the RNA protectant may be included in a medium that further includes a chelator (e.g., EDTA), a buffer (e.g., sodium citrate, sodium acetate, potassium citrate, or potassium acetate, preferably sodium acetate), and/or buffered to a pH between about 4-8 (e.g., about 5, 6, 7). In some embodiments, the cellular tissue is treated with one or more RNA protectants before, contemporaneously with, or after permeabilization. For example, a tissue section can be treated with one or more RNA protectants prior to treatment with one or more permeabilization reagents (e.g., one or more proteases). In another example, a tissue section is treated with a solution including one or more RNA protectants and one or more permeabilization reagents (e.g., one or more proteases). In yet another example, a tissue section is treated with one or more RNA protectants after the tissue section has been treated with one or more permeabilization reagents (e.g., one or more proteases). In some embodiments, a tissue section is treated with one or more RNA protectants prior to fixation. In some embodiments, identifying the location of a captured analyte in the cellular tissue includes a nucleic acid extension reaction. In some embodiments where a capture probe captures a fragmented genomic DNA molecule, a nucleic acid extension reaction includes DNA polymerase. For example, a nucleic acid extension reaction includes using a DNA polymerase to extend the capture probe that is hybridized to the captured analyte (e.g., fragmented genomic DNA) using the captured analyte (e.g., fragmented genomic DNA) as a template. Any DNA polymerase that can extend the capture probe using the captured analyte as a template can be used. Non-limiting examples of DNA polymerases include T7 DNA polymerase; Bsu DNA polymerase; and E. coli DNA Polymerase pol I.

In order to optimize the reagent type, the tissue sections on the TO slide can be treated with different permeabilization reagents, such as at least two, or at least three, or at least four, or at least five, or at least six, or at least seven, or at least eight different reagents or reagent combinations. The tissue sections can be permeabilized using a set of conditions that is constant except for the variations in permeabilization reagent, in order to determine the optimal permeabilization reagent for a cellular tissue. This procedure can be repeated using several additional TO slides to evaluate many different reagents and reagent combinations, in order to further optimize the permeabilization reagent type. Once the optimal reagent is determined, the method can be repeated using various amounts and concentrations of the reagent in order to determine the optimal reagent concentration.

### Fixing Agents

Another parameter that can affect permeabilization is whether or not the cellular tissue is fixed before permeabilization and, if so, the type of fixing agent used to fix the cellular tissue before permeabilization. A fixing agent is a chemical species that preserves the tissue cells and/or morphology before and during permeabilization. For example, an organic solvent such as an alcohol (e.g., ethanol or methanol), ketone (e.g., acetone), or aldehyde (e.g., formaldehyde or glutaraldehyde), or any combination thereof may act as a fixing agent. Alternatively, or in addition, a cross-linking agent may act as a fixing agent. In some cases, a fixing agent may comprise disuccinimidyl suberate (DSS), dimethylsuberimidate (DMS), formalin, and dimethyladipimidate (DMA), dithio-bis (-succinimidyl propionate) (DSP), disuccinimidyl tartrate (DST), and/or ethylene glycol bis (succinimidyl succinate) (EGS), and any combinations thereof. In order to optimize the fixing agent, the tissue sections on the TO slide can be treated with different fixing agents, such as at least two, or at least three, or at least four, or at least five, or at least six, or at least seven, or at least eight different fixing agents. The tissue sections can be permeabilized using a set of conditions that is constant except for the variations in fixing agent, in order to determine the optimal fixing agent for a tissue section.

### Staining Agents

The staining agent (if any) used to stain the tissue section before permeabilization can also influence the level of permeabilization of the tissue section. Staining is typically performed after fixing the cellular tissue and facilitates visualization of the tissue sections, pre and post permeabilization. A wide variety of staining agents can be used, including without limitation acridine orange, Bismarck brown, carmine, Coomassic blue, cresyl violet, 4,6-diamidino-2-phenylindole (DAPI), eosin, hematoxylin, ethidium bromide, acid fuchsine, iodine, methyl green, bisbenzimides, iodine, methyl green, methylene blue, neutral red, Nile blue, Nile red, osmium tetroxide, propidium iodide, rhodamine, safrain, radioisotopes, fluorophores, chemiluminescent compounds, bioluminescent compounds, and combinations thereof.

In order to optimize the staining agent, the tissue sections on the TO slide can be treated with different staining agents, such as at least two, or at least three, or at least four, or at least five, or at least six, or at least seven, or at least eight different staining agents. The tissue sections can be permeabilized using a set of conditions that is constant except for the variations in staining agent, in order to determine the optimal staining agent for a cellular tissue.

### Creating cDNA Footprints of Permeabilized Tissue Sections

Prior to placing the tissue sections on the TO slide, in some examples, each array area of the TO slide includes a plurality of capture probes that comprise capture domains that interact with the mRNA from the corresponding tissue section to cause hybridization, followed by extension of the capture domain, using the hybridized mRNA as a template, resulting in the formation of a cDNA footprint of the tissue section on the TO slide. A description of one suitable cDNA extension protocol is provided in the Visium Spatial Gene Expression Reagent Kits-Tissue Optimization User Guide, published by 10X Genomics, which is incorporated herein by reference. The incorporation of one or more fluorescently labelled dNTPs during reverse transcription results in the production of fluorescent cDNA (i.e., fluorescent cDNA synthesis) which can be detected via fluorescence microscopy, for example.

Other methods of detecting the amount of an analyte, for example mRNA, released from tissue sections overlaid onto an array after cell permeabilization may be used. In some examples, fluorescent probes that hybridize to captured RNAs released from cells may be applied and detected. In some examples, fluorescent probes that hybridize to captured RNAs may prime synthesis of cDNA and be used for detection. Labels other than fluorescent labels may be used (e.g., colorimetric labels).

In one embodiment, a reverse transcription primer includes a branched mRNA-capturing probe having a plurality of hybridization regions arranged in a straight, stepped or branched chain. The hybridization regions serve as capture domains for target mRNA present on the adjacent surface of the tissue sections. Each capture domain can be an oligonucleotide, a polypeptide, a small molecule, or any combination thereof, that binds, captures and/or detects a target mRNA.

Each capture domain can be a functional nucleic acid sequence configured to interact with the target mRNA molecules. The functional sequence can include a poly(T) sequence, which poly(T) sequences are configured to interact with the mRNA molecules via the poly(A) tail of an mRNA transcript.

Capture domains can include ribonucleotides and/or deoxyribonucleotides as well as synthetic nucleotide residues that can participate in Watson-Crick type or analogous base pair interactions. The capture domains can prime a reverse transcription reaction to generate cDNA that is complementary to the captured mRNA molecules. The capture domains can be ligated to one strand of the captured mRNA molecules. For example, SplintR ligase along with RNA or DNA sequences (e.g., degenerate RNA) can be used to ligate a single-stranded RNA to a capture domain. In some embodiments, ligases with RNA-templated ligase activity, e.g., SplintR ligase, T4 RNA ligase 2 or KOD ligase, can be used to ligate a single-stranded mRNA to the capture domain.

In some embodiments, a capture domain includes a splint oligonucleotide. A capture domain can be located at the end of the capture probe and can include a free 3′ end that can be extended, e.g., by template dependent polymerization, to form an extended capture probe. In some embodiments, the capture domain includes a nucleotide sequence that is capable of hybridizing to mRNA present in the cells of the biological tissue contacted with the array. The capture domain can be selected or designed to bind selectively or specifically to a target mRNA by way of hybridization to the mRNA poly(A) tail or other region in the mRNA. Thus, the capture domain can include a poly(T) DNA oligonucleotide, e.g., a series of consecutive deoxythymidine residues linked by phosphodiester bonds, which is capable of hybridizing to the poly(A) tail of mRNA. The capture domain can include nucleotides that are functionally or structurally analogous to a poly(T) tail, for example, a poly(U) oligonucleotide or an oligonucleotide including deoxythymidine analogues. The capture domain can have a sequence that is capable of binding to mRNA. For example, the capture domain can include a nucleic acid sequence (e.g., a poly(T) sequence) capable of binding to a poly(A) tail of an mRNA. In some embodiments, a homopolymer sequence is added to an mRNA molecule using a terminal transferase enzyme in order to produce a molecule having a poly(A) or poly(T) sequence. For example, a poly(A) sequence can be added to an mRNA, thereby making the mRNA capable of capture by a poly(T) capture domain.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located at 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence.

In some embodiments, a capture domain can be semi-random or fully fixed or defined sequence.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes a poly(T)-like sequence and the capture domain of one or more capture probes includes random sequences. In some embodiments, a pool of two or more capture probes form a mixture where the capture domain of one or more capture probes includes a poly(T)-random sequence and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a gene sequence, a motif sequence or common/conserved sequence that it is designed to capture (i.e., a sequence-specific capture domain). Thus, the capture domain can be capable of binding selectively to a desired sub-type or subset of nucleic acid, for example a type or subset of mRNA. In some embodiments, a capture domain includes an “anchor” or “anchoring sequence,” which is a sequence of nucleotides designed to ensure that the capture domain hybridizes to the intended mRNA. The anchor sequence can include a sequence of nucleotides, including a 1-mer, 2-mer, 3-mer or longer sequence. The sequence can be random. For example, a capture domain including a poly(T) sequence can be designed to capture an mRNA. An anchoring sequence can include a random 3-mer (e.g., GGG) that helps ensure that the poly(T) capture domain hybridizes to an mRNA. In some embodiments, an anchoring sequence can be VN, N, or NN (wherein V is A, C or G and N is an nucleotide A, C, G, Tor U). Alternatively, the sequence can be designed using a specific sequence of nucleotides. In some embodiments, the anchor sequence is at the 3′ end of the capture domain. In some embodiments, the anchor sequence is at the 5′ end of the capture domain.

### Quantification of Image Properties

In order to optimize the values of each of the foregoing parameters for permeabilization optimization, the permeabilized tissue sections are removed from the TO slides following hybridization and cDNA synthesis, leaving the cDNA synthesis products as a footprint of the captured and extended mRNA on the array slide. Photomicrographic images are taken of the hybridized cDNA synthesis products and the images are quantified in a manner that is representative of the permeabilization treatment of each tissue section. The two image properties that are of primary interest are image brightness and image sharpness. Brighter images and sharper images generally indicate higher levels of permeabilization.

However, the brightest images and the sharpest images do not always occur with the same set of permeabilization conditions. In such circumstances, it may be advantageous to determine the separate permeabilization conditions that yield the brightest images and the sharpest images, and then determine a set of conditions that achieves an optimal balance of both brightness and sharpness.

In one embodiment, Fast Fourier Transform (FFT) technology can be used to quantify the brightness and sharpness of the photomicrographic images. FFT is an implementation of Discrete Fourier Transform (DFT) that can be applied to convert two-dimensional digital images from the TO array slide image domain to the frequency domain. DFT is a sampled Fourier transform that does not contain all frequencies forming an image but contains a set of frequencies large enough to mathematically describe the image from the TO array slide. FFT decomposes the digital image into its real and imaginary (sine and cosine) components that represent the image in the frequency domain. The number of frequencies in the frequency domain is equal to the number of pixels in the domain image from the TO array slide. For a square image of dimensions N x N, the two-dimensional DFT is represented by the following equation:

$$F ( k , l ) = {\sum }_{i = 0}^{N - 1} {\sum }_{j = 0}^{N - 1} f ( i , j ) {e}^{- ι2π ( \frac{ki}{N} + \frac{lj}{N} )}$$

In the foregoing equation, f (i, j) is the image in the spatial domain and the exponential function is the base function corresponding to each point F (k, 1) in the Fourier space. In simple terms, the value of each point F (k, 1) in the Fourier space is calculated by multiplying the TO image with the corresponding base function and summing the results. The basis functions are sine and cosine waves with increasing frequencies. F (0, 0) represents the DC component of the image which corresponds to the average brightness. F (N−1, N−1) represents the highest frequency and is indicative of image sharpness.

The FFT simplifies the foregoing calculations using mathematical techniques that separate the two-dimensional transform into two one-dimensional transforms, one on the horizontal direction followed by the other in the vertical direction based on the result of the horizontal transform. Yet even with this simplification, the one-dimensional FFT still has N² complexity. The FFT further simplifies the calculation by reducing the N² complexity to N log₂N. The FFT thus restricts the size of the image that can be transformed, often to N=2ⁿ where n is an integer.

The FFT produces a complex numerical-valued output image that can be displayed with either real and imaginary (sine and cosine) parts or with magnitude and phase.

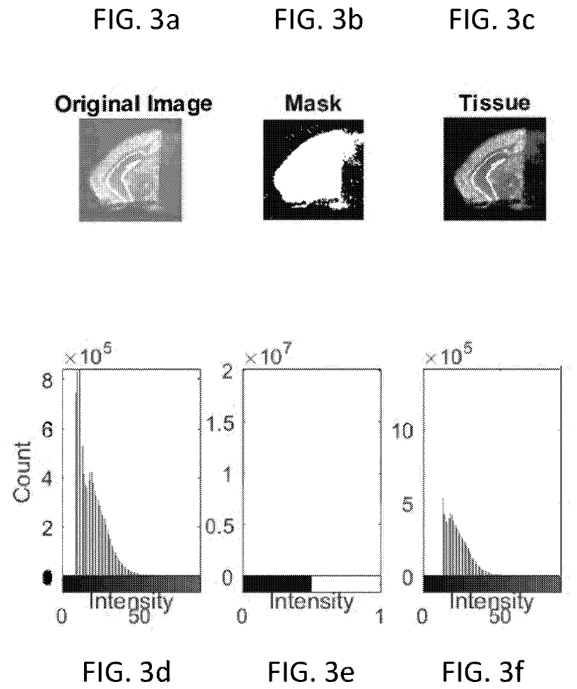

Again, image magnitude is typically indicative of brightness, while image phase inversely correlates with frequency, which is indicative of image resolution or sharpness. FIGS. 3a-3c illustrate one technique for quantifying image properties. FIG. 3a is a representative photomicrographic image of a cDNA footprint taken from permeabilized mouse brain tissue. This image has both a tissue region and a background region (region around the tissue). FIG. 3b represents a mask image in which the tissue region is assigned pixel values of 1 and the background region is assigned pixel values of zero. The mask can be obtained using various techniques for screening out the background, such as by using an intensity threshold for brightness or fluorescence of the image. FIG. 3c is a tissue image obtained by merging the original and mask images of FIGS. 3a and 3b together. In FIG. 3c, the tissue pixel values remain intact while the background pixel values are reduced to zero.

FIGS. 3d-3f are histograms of pixel count versus pixel intensity for the images in FIGS. 3a-3c, respectively. The histograms provide one method for quantifying the intensity of the images, which is representative of image brightness. The image brightness is indicative of the sensitivity of the assay to permeabilization. The FFT histograms in FIGS. 3d-3f quantify the image intensity for one permeabilized tissue section that was permeabilized using one set of parameters (one value for temperature, time, permeabilization reagent, etc.). By varying one or more of the foregoing permeabilization parameters, images like FIGS. 3a-3c and histograms like FIGS. 3d-3f can be generated to determine which value of each permeabilization parameter or combination of parameters yields the optimal brightness of the image, and therefore the optimal permeabilization condition based on optimal brightness.

The images and histograms can be generated from cDNA footprints representing a wide variety of different permeabilization conditions for a tissue type. The resulting plots and data library can then be used to correlate the image property (in this case, image brightness) with the variations in the parameter (permeabilization temperature, time, reagent type and concentration, fixing and staining agents, etc.) to determine a value for the parameter that yields an optimal level of permeabilization. The same quantification procedure can be used when evaluating different parameters and combinations of parameters (permeabilization temperature, reagents, fixing and staining agents, etc.) to determine the optimal values for each parameter and for any combination of parameters. Moreover, different metrics can be generated from the histograms, including without limitation maximum image intensity or rightness, average image intensity or brightness, and median image intensity or brightness. As explained above, iterative techniques can be employed to optimize the values of two or more parameters simultaneously.

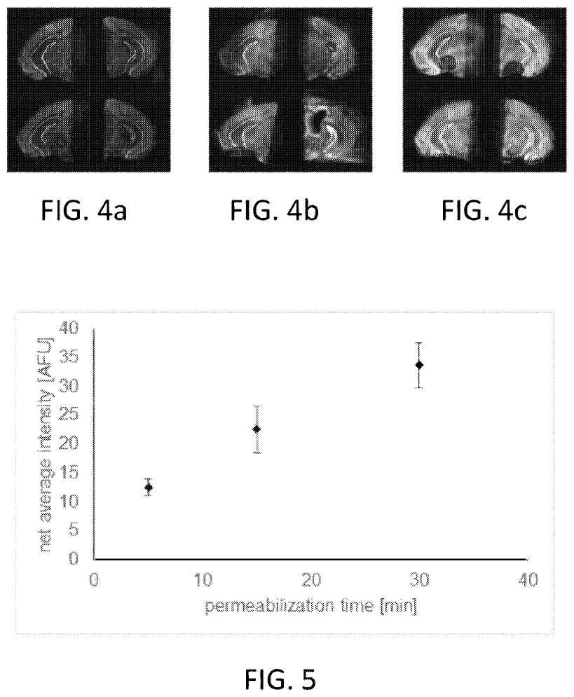

FIGS. 4a-4c arc photomicrographic images representing cDNA footprints of mouse brain tissue that has been permeabilized for 5 minutes, 15 minutes and 30 minutes, respectively, using the permeabilization conditions described above (37° C. permeabilization, methanol fixed with no staining, protease K and 10% sodium dodecyl sulfate reagents). FIG. 5 is a FFT plot of net average image intensity (AFU) versus permeabilization time. The quantitative image analysis shown in FIG. 5 is consistent with the photomicrographs (FIGS. 4a-4c) which clearly show a brighter image at 30 minutes than at 15 minutes, and a brighter image at 15 minutes than at 5 minutes.

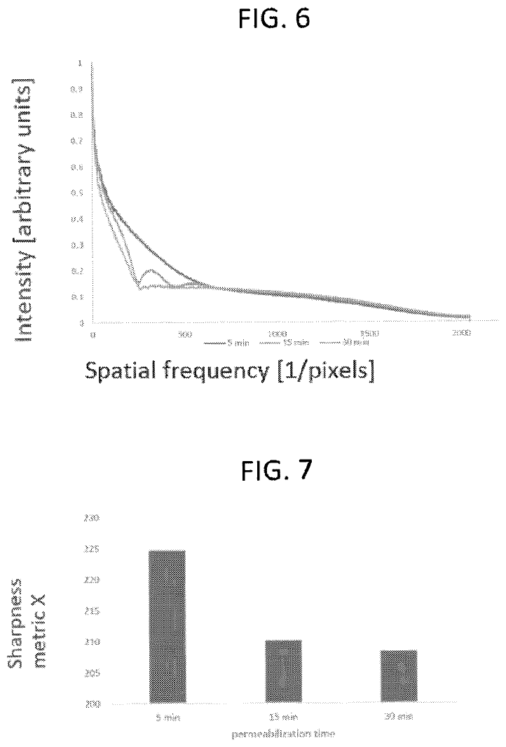

FFT images can also be used to quantify spatial frequency (frequency of sine waves over a range), which is a measure of image sharpness. This can be accomplished by processing the FFT image to extract a radial profile or a power spectrum from the FFT image. FIG. 6 includes exemplary radial profiles showing the image intensity (AFU's) versus spatial frequency (measured in units of l/pixel length), for each of the three photomicrographs shown in FIGS. 4a-4c. The curves largely coincide except for the frequency range of about 100 to about 500 reciprocal pixels. Within that range, the image intensity of the cDNA footprint of the tissue section permeabilized for 5 minutes (upper curve) exceeds the image intensity of the cDNA footprint of the tissue section permeabilized for 15 minutes (middle curve), which in turn exceeds the image intensity of the cDNA footprint of the tissue section permeabilized for 30 minutes (lower curve). For each of the images, as the spatial frequency increases, the image intensity decreases. However, the noted difference in image intensities in the 100-500 reciprocal pixel rage indicates an overall sharper image resulting at 5 minutes permeabilization time.

FIG. 7 is a plot of a selected sharpness metric versus permeabilization time. In this instance, the selected sharpness metric is average spatial frequency over a selected range. The average spatial frequency, which is indicative of image sharpness, is higher at the lower permeabilization time of 5 minutes than at the higher permeabilization times of 15 and 30 minutes. This is consistent with the information derived from FIG. 6.

The foregoing underscores the importance of considering both the image brightness and the image sharpness when determining optimal permeabilization conditions for a tissue section. Individual consideration of one image property to the exclusion of the other may fail to reveal that the optimal conditions for image brightness may not be the same as the optimal conditions for image sharpness. The FFT calculations reveal that the images shown in FIGS. 4a-4c become less sharp as they become brighter, as the permeabilization time is increased.

The FFT plots can be used to calculate a variety of metrics that are representative of an image property, including without limitation average frequency over a selected range of frequencies, average intensity over a selected range of frequencies, and ratio of frequencies over a plurality of selected ranges. The quantitative method of the invention can be used to optimize tissue section permeabilization for one parameter (e.g., temperature, time, reagent type and concentration, fixation agent, staining agent), for two or more parameters in sequence, for three or more parameters in sequence, for four or more parameters in sequence, or for five or more parameters in sequence. Using iterative techniques as described above, the quantitative method of the invention can also be used to optimize the permeabilization for two or more parameters simultaneously, for three or more parameters simultaneously, for four or more parameters simultaneously, or for five or more parameters simultaneously. For each of the permeabilization parameters and combinations of parameters, the method of the invention can be used to achieve an optimum image brightness, and optimum image sharpness, an optimum combination of image brightness and image sharpness, or any other image property or combination of image properties.

### Additional Quantification Techniques and Examples

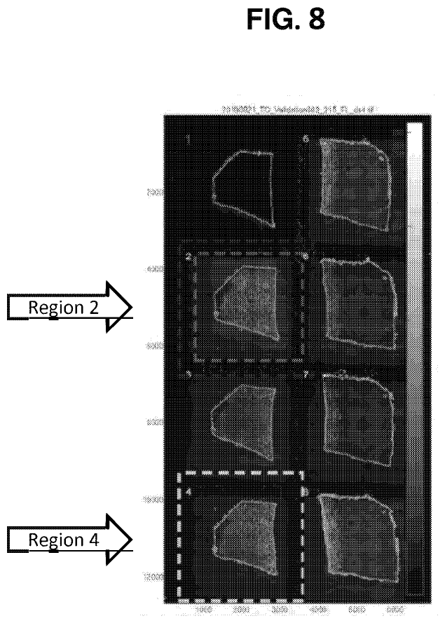

FIGS. 8-34 depict example results of additional techniques for quantifying image properties resulting from cDNA footprints of exemplary permeabilized tissue samples. Fixed, frozen tissue sections were placed on tissue optimization slides divided into cight rectangular capture regions 1-8, as described above with respect to FIG. 1. The permeabilization times for the tissue samples placed in each of the capture regions 1-8 were as follows: Region 1—negative control (no permeabilization), Region —3 minutes, Region 3—6 minutes, Region 4—12 minutes, Region 5—18 minutes, Region 6—24 minutes, Region 7—30 minutes, Region 8—36 minutes. For each example, the tissue sample was prepared, fixed, permeabilized, mRNA hybridized and fluorescently labelled cDNA synthesis products imaged using the procedure set forth in the User Guide for Visium Spatial Gene Expression Reagent Kits—Tissue Optimization, published by 10X Genomics, which is incorporated herein by reference, the permeabilization variable that was optimized was time. FIG. 8 provides photomicrographic images of a cDNA footprint representing

permeabilization of human kidney tissue for the eight different times. The fluorescent signals were qualitatively ranked and the cDNA footprint in Region 4, representing 12 minutes permeabilization, was chosen as producing the brightest and sharpest image. However, quantitative analysis of the images revealed that the cDNA footprint in Region 2, representing 3 minutes permeabilization, produced both the brightest and the sharpest image.

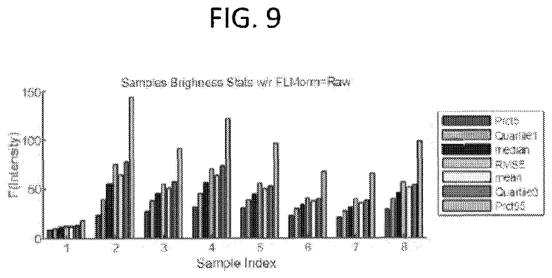

FIG. 9 is a bar graph showing pixel intensity distribution (measured in arbitrary fluorescent units) for the eight images from the tissue optimization slide represented in FIG. 8. The plots were generated from image information obtained using an inverted fluorescent microscope. The bars in each group (x-axis; Sample Indices 1-8) in the bar graph, from left to right in each group, indicate 5^{th} percentile, 25^{th} percentile, 50^{th} percentile, root mean square, mean, 75^{th} percentile and 95^{th} percentile. As shown in FIG. 9, the cDNA footprint from Region 2, representing 3 minutes permeabilization, produced the highest overall image intensity, representing the brightest image. Interestingly, the cDNA footprint from Region 4, representing 12 minutes permeabilization, produced the second highest overall image intensity, whereas the cDNA footprint from Region 3, representing 6 minutes permeabilization, produced less overall image intensity that the cDNA footprints from Regions 2 and 4.

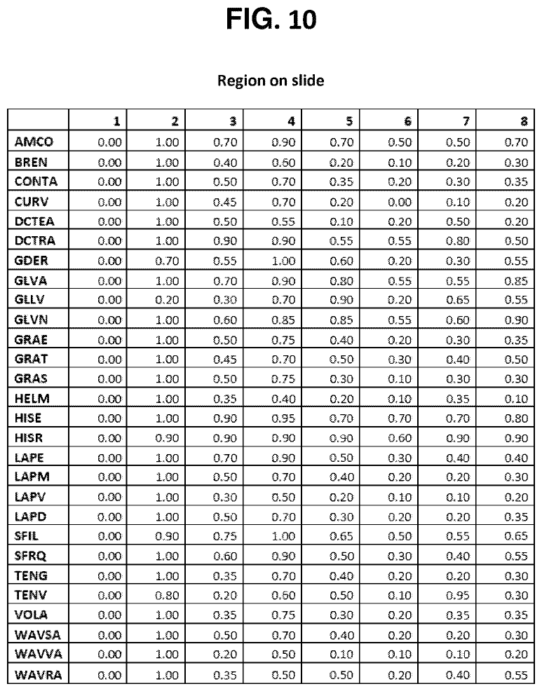

FIG. 10 is a table containing normalized metrics for the eight images (columns 1-8) from the tissue optimization slide represented in FIG. 8. The metrics normalize image sharpness on a scale of zero to 1 with 1 representing the sharpest image, using 28 image sharpness algorithms (rows AMCO through WAVRA in the table). The algorithms are indicated by acronyms in FIG. 10, which have meanings according to the following Table 1. The procedures for measuring each algorithm are explained in the indicated references, each of which is public and incorporated herein by reference.

TABLE 1
Algorithms for Quantifying Image Sharpness

| Acronym | Name | Reference |
|---|---|---|
| ACMO | Absolute Central | Shirvaikar, “An Optical Measure for Camera Focus |
| | Moment | and Exposure,” Proceedings of the IEEE SSST (2004) |
| BREN | Brenner's function | Santos et al., “Evaluation of Autofocus Functions in |
| | | Molecular Cytogenetic Analysis,” J. Microscopy, Vol. |
| | | 188, pt. 3 (December 1997), pp. 264-272 |
| CONT | Image contrast | Nanda et al. “Practical calibrations for a Real-Time |
| | | Digital Omnidirectional Camera,” Proceedings of |
| | | CVPR, Technical Sketch (January 2001) |
| CONTA | Image contrast A | Nanda et al., “Practical calibrations for a Real-Time |
| | | Digital Omnidirectional Camera,” Proceedings of |
| | | CVPR, Technical Sketch (January 2001) |
| CURV | Image curvature | Helmli et al., “Adaptive Shape from Focus with An |
| | | Error Estimation in Light Microscopy,” IEEE |
| | | Conference Publication, ISPA (2001) |
| DCTE | DCT energy ratio | Shen et al., “Robust Focus Measure for Low Contrast |
| | | Images,” IEEE Digest of Technical Papers, |
| | | International Conference on Consumer Electronics |
| | | (2006) |
| DCTR | DCT reduced energy | Lee et al., “Reduced Energy Measure Ratio for Robust |
| | ratio | Autofocusing in Digital Camera,” IEEE Signal |
| | | Processing Letters, Vol. 16, Issue 2 (February 2009) |
| DCTEA | DCT energy ratio A | Shen et al., “Robust Focus Measure for Low Contrast |
| | | Images,” IEEE Digest of Technical Papers, |
| | | International Conference on Consumer Electronics |
| | | (2006) |
| DCTRA | DCT reduced energy | Lee et al., “Reduced Energy Measure Ratio for Robust |
| | ratio A | Autofocusing in Digital Camera,” IEEE Signal |
| | | Processing Letters, Vol. 16, Issue 2 (February 2009) |
| GDER | Gaussian derivative | Geusebroek et al., “Robust Autofocusing in |
| | | Microscopy,” Cytometry 39: 109 (2000) |
| GLVA | Graylevel variance | Krotkov et al., “Range from Focus,” 1986 IEEE |
| | | Conference, Vol. 3, pp. 1093-1098 (1986) |
| GLLV | Graylevel local | Pech-Pacheco et al., “Diatom Autofocusing in |
| | variance | Brightfield Microscopy: A Comparative Study,” IEEE |
| | | Proceedings 15^{th} International Conference on Pattern |
| | | Recognition, ICPR-2000 (2000) |
| GLVN | Normalized GLV | Santos et al., “Evaluation of Autofocus Functions in |
| | | Molecular Cytogenetic Analysis,” J. Microscopy, Vol. |
| | | 188, pt. 3 (December 1997), pp. 264-272 |
| GRAE | Energy of gradient | Subbarao et al., “Focusing Techniques,” SPIE |
| | | Proceedings, Vol. 1823, Machine Vision Applications, |
| | | Architectures, and Systems Integration (Nov. 1, |
| | | 1992) |
| GRAT | Thresholded gradient | Santos et al., “Evaluation of Autofocus Functions in |
| | | Molecular Cytogenetic Analysis,” J. Microscopy, Vol. |
| | | 188, pt. 3 (December 1997), pp. 264-272 |
| GRAS | Squared gradient | Eskicioglu et al., “Image Quality Measures and Their |
| | | Performance,” Computer Science, IEEE Trans. |
| | | Communications (1995) |
| HELM | Helmli's mean | Helmli et al., “Adaptive Shape from Focus with An |
| | method | Error Estimation in Light Microscopy,” IEEE |
| | | Conference Publication, ISPA (2001) |
| HISE | Histogram entropy | Krotkov et al., “Range from Focus,” 1986 IEEE |
| | | Conference, Vol. 3, pp. 1093-1098 (1986) |
| HISR | Histogram range | Firestone et al., Comparison of Autofocus Methods |
| | | for automated Microscopy,” cytometry 12: 195-206 |
| | | (1991) |
| LAPE | Energy of Laplacian | Subbarao et al., “Focusing Techniques,” SPIE |
| | | Proceedings, Vol. 1823, Machine Vision Applications, |
| | | Architectures, and Systems Integration (Nov. 1, |
| | | 1992) |
| LAPM | Modified Laplacian | Nayar, “Shape from Focus,” CMU-RI-TR-89-27, |
| | | Carnegie Mellon University (1989) |
| LAPV | Variance of | Pech-Pacheco et al., “Diatom Autofocusing in |
| | Laplacian | Brightfield Microscopy: A Comparative Study,” IEEE |
| | | Proceedings 15^{th} International Conference on Pattern |
| | | Recognition, ICPR-2000 (2000) |
| LAPD | Diagonal Laplacian | Thelen et al., “Improvements in Shape-from-Focus for |
| | | Holographic Reconstructions,” IEEE Transactions on |
| | | Image Processing, Vol. 18, No. 1, pp. 151-157 (2009) |
| SFIL | Steerable filters | Minhas et al., “3D Shape from Focus and Depth Map |
| | | computation Using Steerable Filters,” ICIAR 2009: |
| | | Image Analysis and Recognition, pp. 573-583 (2009” |
| SFRQ | Spatial frequency | Eskicioglu et al., “Image Quality Measures and Their |
| | | Performance,” Computer Science, IEEE Trans. |
| | | Communications (1995) |
| TENG | Tenengrad | Krotkov et al., “Range from Focus,” 1986 IEEE |
| | | Conference, Vol. 3, pp. 1093-1098 (1986) |
| TENV | Tenengrad variance | Pech-Pacheco et al., “Diatom Autofocusing in |
| | | Brightfield Microscopy: A Comparative Study,” IEEE |
| | | Proceedings 15^{th} International Conference on Pattern |
| | | Recognition, ICPR-2000 (2000) |
| VOLA | Vollath’s correlation | Santos et al., “Evaluation of Autofocus Functions in |
| | | Molecular Cytogenetic Analysis,” J. Microscopy, Vol. |
| | | 188, pt. 3 (December 1997), pp. 264-272 |
| WAVS | Sum of Wavelet | Yang et al., “Wavelet-Based Autofocusing and |
| | coefficients | Unsupervised Segmentation of Microscopic Images,” |
| | | IEEE Xplore Conference: Intelligent Robots and |
| | | Systems, Vol. 3 (2003) |
| WAVSA | Sum of Wavelet | Yang et al., “Wavelet-Based Autofocusing and |
| | coefficients A | Unsupervised Segmentation of Microscopic Images,” |
| | | IEEE Xplore Conference: Intelligent Robots and |
| | | Systems, Vol. 3 (2003) |
| WAVV | Variance of Wavelets | Yang et al., “Wavelet-Based Autofocusing and |
| | | Unsupervised Segmentation of Microscopic Images,” |
| | | IEEE Xplore Conference: Intelligent Robots and |
| | | Systems, Vol. 3 (2003) |
| WAVR | Multi-level two- | Gonzalez et al., “Digital Image Processing Using |
| | dimensional inverse | MATLAB,” Prentice-Hall, 2004 Revision |
| | FWT | |
| WAVRA | Multi-level two- | Gonzalez et al., “Digital Image Processing Using |
| | dimensional inverse | MATLAB,” Prentice-Hall, 2004 Revision |
| | FWT A | |

As shown in FIG. 10, the “Region 2” (sample 2) cDNA footprint (3 minutes permeabilization time) yielded the best image sharpness in 23 of the 28 algorithms. The “Region 4” (sample 4) cDNA footprint (12 minutes permeabilization time) yielded the second-best image sharpness according to most of the algorithms.

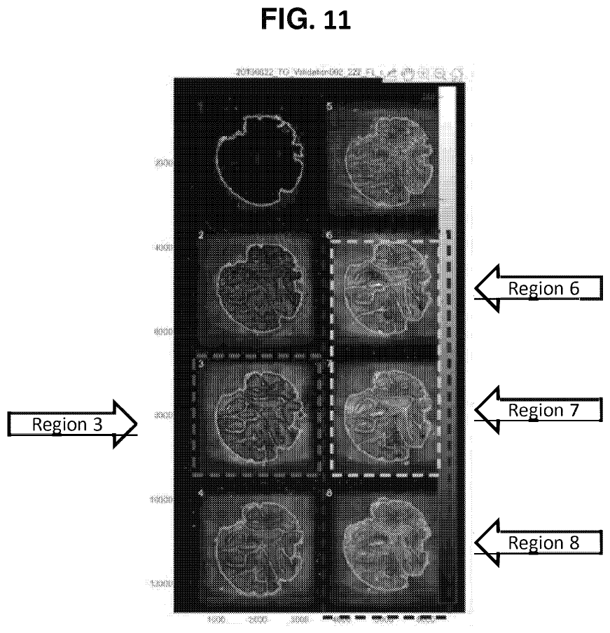

FIG. 11 provides photomicrographic images of cDNA footprints resulting from permeabilization of samples of human small intestine tissue for the eight different times: Region 1—negative control (no permeabilization), Region 2—3 minutes, Region 3—6 minutes, Region 4—12 minutes, Region 5—18 minutes, Region 6—24 minutes, Region 7—30 minutes, Region 8—36 minutes. Each tissue sample was prepared, fixed, permeabilized, hybridized and imaged using the procedure set forth in the above-identified User Guide for Visium Spatial Gene Expression Reagent Kits—Tissue Optimization, time was again the permeabilization variable.

Referring to FIG. 11, the eight permeabilization conditions were qualitatively ranked and the images of the cDNA footprints in Regions 6 and 7, representing 24 minutes and 30 minutes permeabilization, respectively, were chosen as producing the brightest and sharpest images. However, quantitative analysis of the images revealed that the cDNA footprint in Region 3, representing 6 minutes permeabilization, produced the sharpest image, and that the cDNA footprint in Regions 6-8, representing 24-36 minutes permeabilization, produced the brightest images.

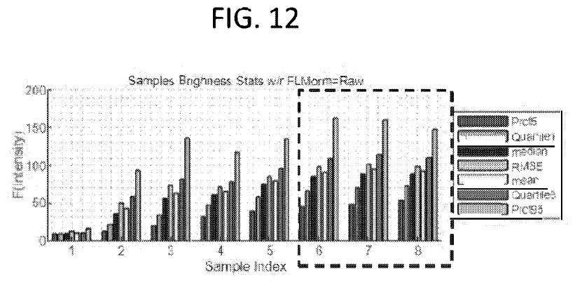

FIG. 12 is a bar graph showing pixel intensity distribution (measured in arbitrary fluorescent units) for the eight images generated in FIG. 11. The plots were generated from image information obtained using an inverted fluorescent microscope. The bars in each group (x-axis; Sample Indices 1-8) in the bar graph, from left to right in each group, indicate 5^{th} percentile, 25^{th} percentile, 50^{th} percentile, root mean square, mean, 75^{th} percentiles and 95^{th} percentile. As shown in FIG. 12, the cDNA footprints in Regions 6 and 7, representing 24 and 30 minutes permeabilization, respectively, produced the greatest overall image intensity, indicating the brightest images. The cDNA footprint in Region 8, representing 36 minutes permeabilization, produced almost as much overall image intensity (brightness) as the cDNA footprints in Regions 6 and 7.

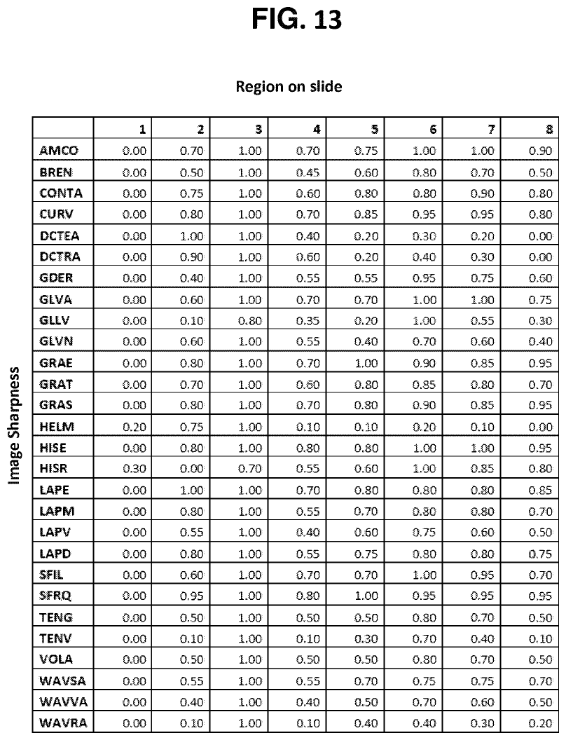

FIG. 13 is a table containing normalized metrics for the eight images (columns 1-8) from the tissue optimization slide represented in FIG. 11, that normalizes image sharpness on a scale of zero to 1 using the 28 image sharpness algorithms described above (rows AMCO through WAVRA in the table). As shown in FIG. 13, the “Region 3” cDNA footprint (6 minutes permeabilization time) produced the highest image sharpness in 26 of the 28 algorithms. The “Region 6” cDNA footprint (24 minutes permeabilization time) produced the second-best image sharpness according to most of the algorithms.

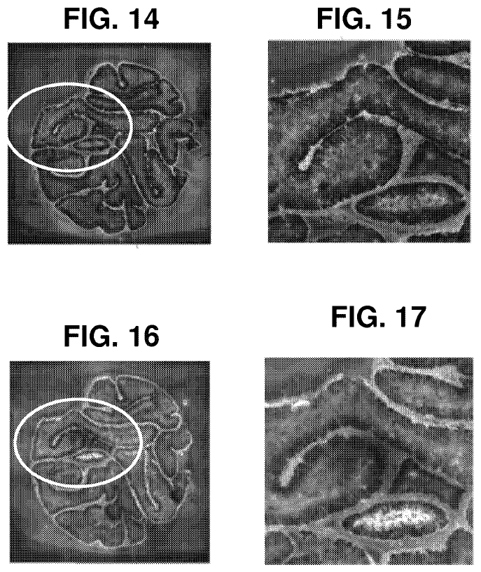

FIGS. 14 and 15 show magnifications of the sharpest image resulting from cDNA footprints of the permeabilized small intestine tissue, taken from Region 3 (6 minutes permeabilization time). FIGS. 16 and 17 show magnifications of the brightest image resulting from cDNA footprints of the permeabilized small intestine tissue, taken from Region 6 (24 minutes permeabilization time). Both the sharpest and the brightest images are useful in visualizing the degree of mRNA and subsequent cDNA synthesis on the tissue optimization slide.

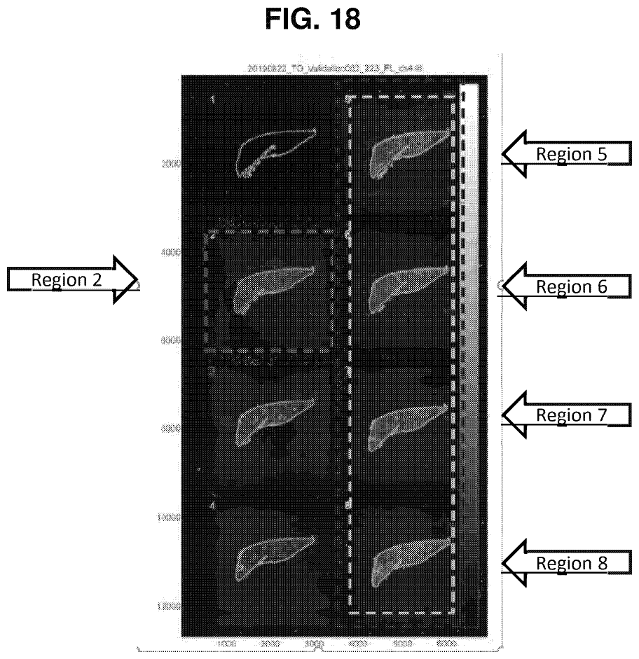

FIG. 18 provides photomicrographic images of cDNA footprints resulting from permeabilization of samples of mouse lung tissue for the eight different times: Region 1—negative control (no permeabilization), Region 2—3 minutes, Region 3—6 minutes, Region 4—12 minutes, Region 5—18 minutes, Region 6—24 minutes, Region 7—30 minutes, Region 8—36 minutes. Each tissue sample was prepared, fixed, permeabilized, hybridized and imaged using the procedure set forth in the above-identified User Guide for Visium Spatial Gene Expression Reagent Kits—Tissue Optimization, the permeabilization time was varied.

Referring to FIG. 18, the eight conditions were qualitatively ranked and the cDNA footprints in Regions 5, 6, 7 and 8, representing 18 minutes, 24 minutes, 30 minutes and 36 minutes permeabilization, respectively, were chosen as producing the brightest and sharpest images. However, quantitative analysis of the images revealed that the cDNA footprint in Region 2, representing 3 minutes permeabilization, produced the sharpest image, and that the tissue samples in Regions 5-8, representing 18-36 minutes permeabilization, produced the brightest images.

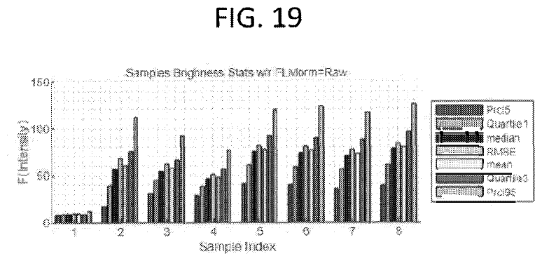

FIG. 19 is a bar graph showing pixel intensity distribution (measured in arbitrary fluorescent units) for the eight images represented in FIG. 18. The plots were generated from image information obtained using an inverted fluorescent microscope. The bars in each group (x-axis; Sample Indices 1-8) in the bar graph, from left to right in each group, indicate 5^{th} percentile, 25^{th} percentile, 50^{th} percentile, root mean square, mean, 75^{th} percentiles and 95^{th} percentile. As shown in FIG. 19, the cDNA footprints in Regions 5, 6, 7 and 8, representing 18, 24, 30 and 36 minutes permeabilization, respectively, produced the greatest overall image intensities, indicating the brightest images. The cDNA footprint in Region 8, representing 36 minutes permeabilization, produced slightly better overall image intensity (brightness) than the cDNA footprints in Regions 5, 6 and 7.

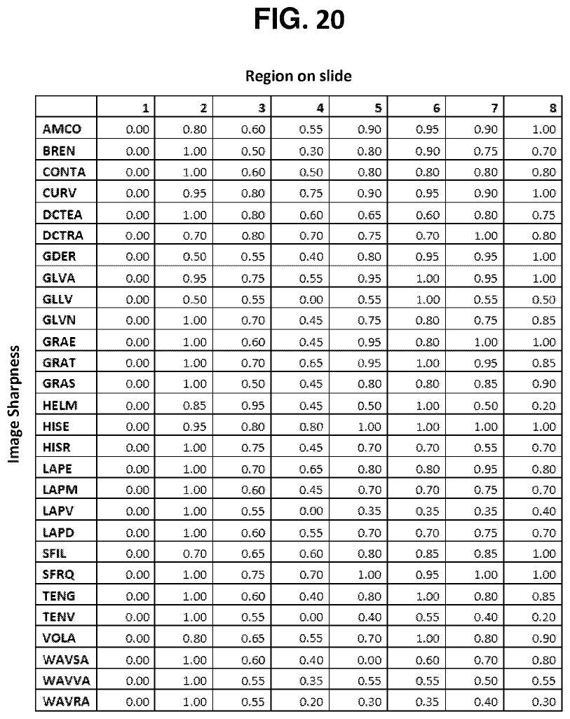

FIG. 20 is a table containing normalized metrics for the eight images (columns 1-8) represented in FIG. 18 that normalizes image sharpness on a scale of zero to 1, measured using the 28 image sharpness algorithms described above (rows AMCO through WAVRA in the table). As shown in FIG. 20, the “Region 2” cDNA footprint (3 minutes permeabilization time) produced the best image sharpness in 19 of the 28 algorithms. The “Region 6” cDNA footprint (24 minutes permeabilization time) produced the second-best image sharpness according to most of the algorithms.

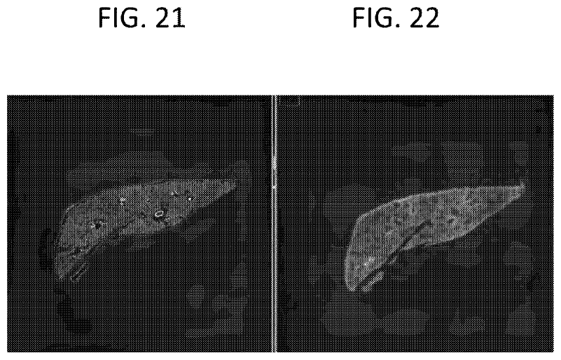

FIGS. 21 and 22 show magnifications of the sharpest image represented in FIG. 18, taken from Region 2 (3 minutes permeabilization time), and one of the brightest images represented in FIG. 18, taken from Region 5 (18 minutes permeabilization time). Again, both the sharpest and the brightest images are useful in visualizing the degree of mRNA capture and subsequent cDNA synthesis on the tissue optimization slide.

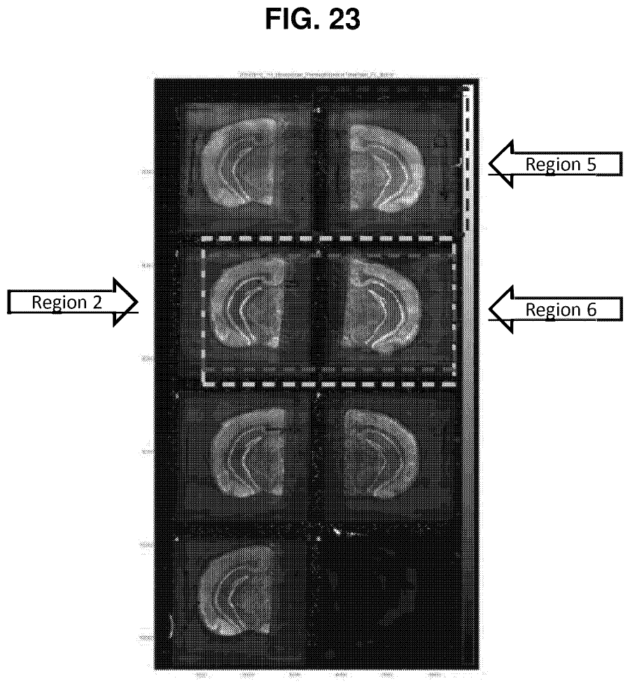

FIG. 23 provides photomicrographic images of cDNA footprints resulting from permeabilization of samples of mouse brain tissue for the eight different times: Region 1—negative control (no permeabilization), Region 2—3 minutes, Region 3—6 minutes, Region 4—12 minutes, Region 5—18 minutes, Region 6—24 minutes, Region 7—30 minutes, Region 8—36 minutes. Each tissue sample was prepared, fixed, permeabilized, hybridized and imaged using the procedure set forth in the above-identified User Guide for Visium Spatial Gene Expression Reagent Kits—Tissue Optimization, permeabilization time was varied.

Referring to FIG. 23, the eight images were qualitatively ranked and the cDNA footprints in Regions 2 and 6, permeabilized for 3 minutes and 24 minutes, respectively, were chosen as producing the brightest and sharpest images. However, quantitative analysis of the images revealed that the cDNA footprint in Region 5, representing 18 minutes permeabilization, produced the brightest image, and that the CDNA footprints in Regions 2 and 6, representing 3 minutes and 24 minutes permeabilization, produced the sharpest images.

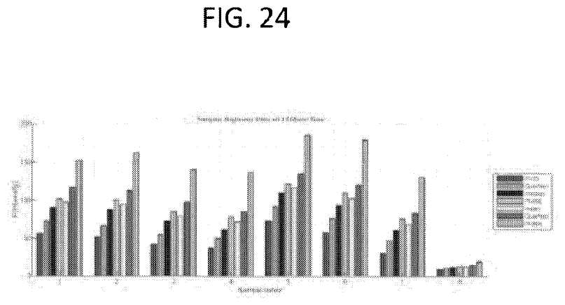

FIG. 24 is a bar graph showing pixel intensity distribution (measured in arbitrary fluorescent units) for the eight images represented in FIG. 23. The plots were generated from image information obtained using an inverted fluorescent microscope. The bars in each group (x-axis; Sample Indices 1-8) in the bar graph, from left to right in each group, indicate 5^{th} percentile, 25^{th} percentile, 50^{th} percentile, root mean square, mean, 75^{th} percentiles and 95^{th} percentile. As shown in FIG. 24, the cDNA footprints in Regions 5, representing 18minutes permeabilization, produced the greatest overall image intensity, indicating the brightest image. The cDNA footprint in Region 6, representing 24 minutes permeabilization, produced a close second-place for image brightness.

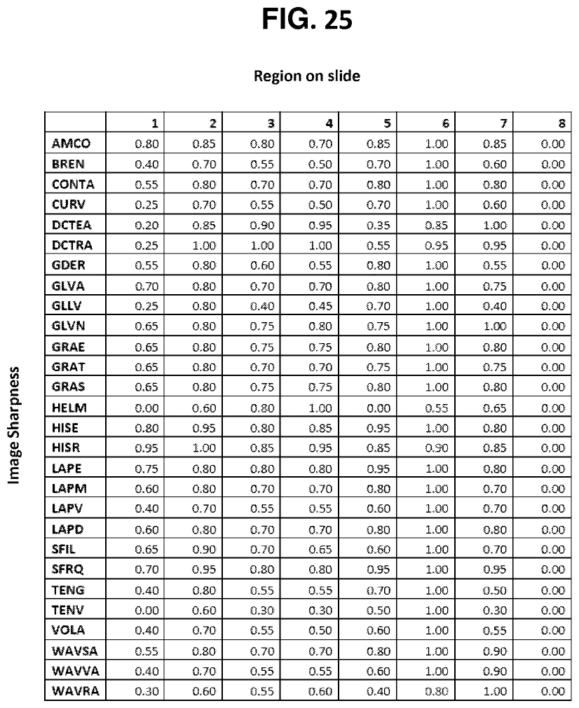

FIG. 25 is a table containing normalized metrics for the eight images (columns 1-8) represented in FIG. 23 that normalizes image sharpness on a scale of zero to 1, measured using the 28 image sharpness algorithms described above (rows AMCO through WAVRA in the table). As shown in FIG. 25, the “Region 6” cDNA footprint (24 minutes permeabilization time) produced the best image sharpness in 24 of the 28 algorithms. The “Region 2” cDNA footprint (3 minutes permeabilization time) produced the second-best image sharpness according to most of the algorithms.

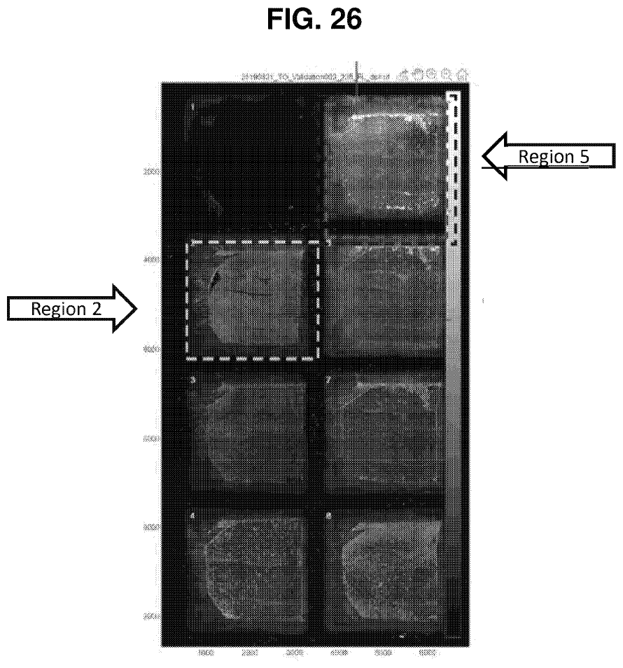

FIG. 26 provides photomicrographic images of cDNA footprints resulting from permeabilization of samples of human liver tissue for the eight different times: Region 1-negative control (no permeabilization), Region 2—3 minutes, Region 3—6 minutes, Region 4—12 minutes, Region 5—18 minutes, Region 6—24 minutes, Region 7—30 minutes, Region 8—36 minutes. Each tissue sample was prepared, fixed, permeabilized, hybridized and imaged using the procedure set forth in the above-identified User Guide for Visium Spatial Gene Expression Reagent Kits—Tissue Optimization, permeabilization time was varied.

Referring to FIG. 26, the eight images were qualitatively ranked and the cDNA footprint in Region 2, representing 3 minutes permeabilization, was chosen as producing the brightest and sharpest image. However, quantitative analysis of the images revealed that the cDNA footprint in Region 5, representing 18 minutes permeabilization, yielded the brightest and the sharpest image.

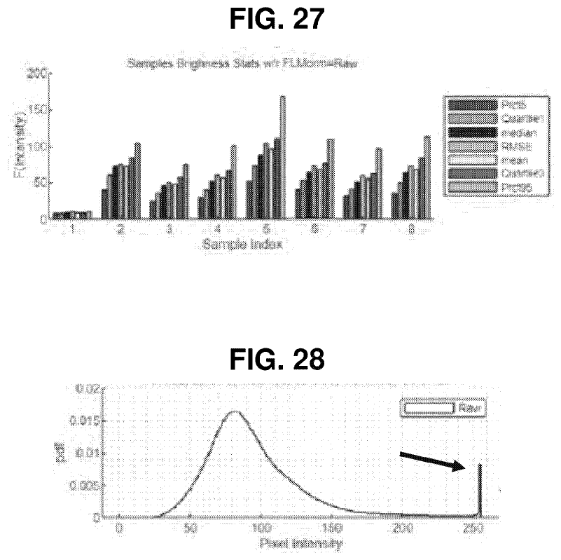

FIG. 27 is a bar graph showing pixel intensity distribution (measured in arbitrary fluorescent units) for the eight images represented in FIG. 26. The plots were generated from image information obtained using an inverted fluorescent microscope. The bars in each group (x-axis; Sample Indices 1-8) in the bar graph, from left to right in each group, indicate 5^{th} percentile, 25^{th} percentile, 50^{th} percentile, root mean square, mean, 75^{th} percentiles and 95^{th} percentile. As shown in FIG. 27, the cDNA footprint in Region 5, representing 18minutes permeabilization, produced the greatest overall image intensity, indicating the brightest image. There was no close second-place image.

FIG. 28 is a histogram of pixel intensity versus pixel count for the brightest image represented in Region 5 of FIG. 26. The arrow points to saturated pixels having an intensity of about 260 arbitrary fluorescent units. The existence of saturated pixels diminishes the reliability of the quantitative image analysis and indicates the need for adjustments to the magnification, filtering, and/or other controls when taking the image.

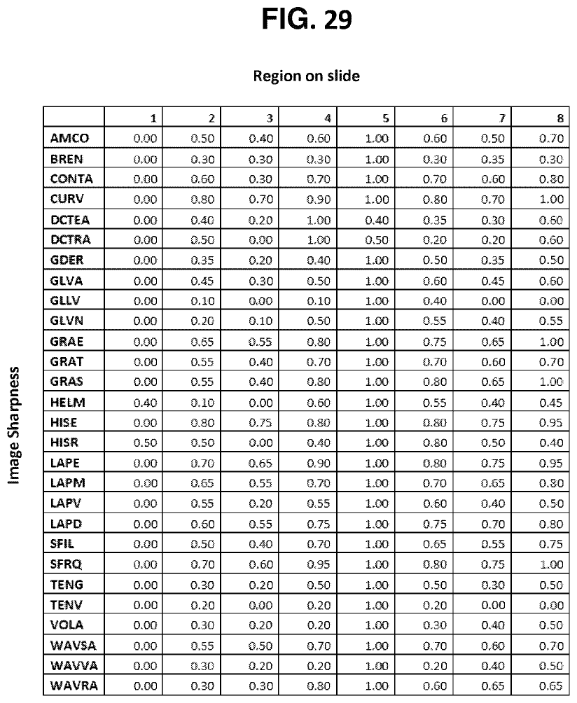

FIG. 29 is a table containing normalized metrics for the eight images (columns 1-8) represented in FIG. 26 that normalizes image sharpness on a scale of zero to 1, measured using the 28 image sharpness algorithms described above (rows AMCO through WAVRA in the table). As shown in FIG. 29, the “Region 5” cDNA footprint (18 minutes permeabilization time) produced the best image sharpness in 26 of the 28 algorithms. There was no close second-place image.

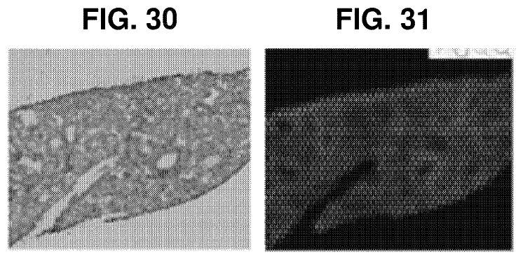

FIGS. 30-34 represent a quantification technique in which an original photomicrographic image of a cDNA footprint of a permeabilized tissue sample could be transformed into a spot image (FIG. 31) for further analysis and quantification. FIGS. 30 and 31 are original and spot images, respectively, of a cDNA footprint of a permeabilized mouse lung tissue section. Transformation of the photomicrographic image into a representative spot image can be accomplished using a variety of known techniques.

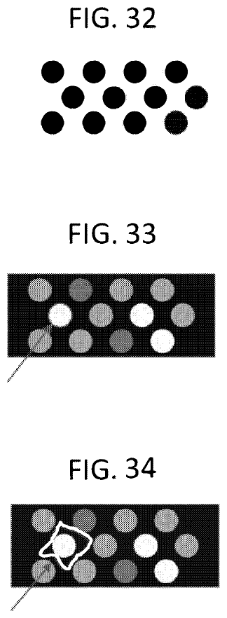

Once the image is transformed, any portion of the spot image can be represented by an array of spots having a size and spacing (pitch), as shown in FIG. 32. In a first embodiment of the spot quantification technique, shown in FIG. 33, the average pixel value (image intensity) of an individual spot, represented by the arrow, can be calculated as the average pixel value of all pixels within each spot. The pixel values of the spaces between the spots are set at zero, and only the average pixel values of the pixels covered by the individual spots are determined. Each spot can thus have an average pixel value that represents the brightness of the portion of the image covered by the spot.

In a second embodiment of the spot quantification technique, shown in FIG. 34, the average pixel value of an individual spot, represented by the arrow, takes into account the average pixel value of both a) the pixels within each spot, and b) the pixels that are outside the spot but within a defined vicinity of the spot (inside the white traced arca). Each spot can be assigned an average pixel value that represents the portions of the image covered by the spot and adjacent to the spot.

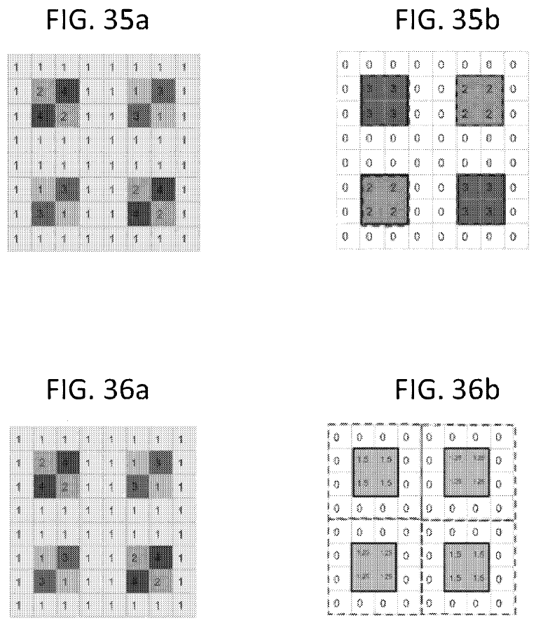

FIGS. 35a and 35b represent one example of pixel averaging according to the first embodiment of the spot quantification technique, based on original and spot images of cDNA footprints of a mouse lung tissue. Referring to FIG. 35a, each spot contained four pixels and had a spot diameter of 50 microns, and the pitch (spacing between spots) averaged 60 microns. Referring to FIG. 35b, only the pixel values within each spot were averaged, while the pixels between the spots were assigned values of zero and not included in the averages. This resulted in an average pixel value of 3 for the upper left spot (the average of 2, 4, 2 and 4), an average pixel value of 3 for the upper right spot (the average of 1, 3, 1, 3), an average pixel value of 2 for the lower left hand spot (the average of 1, 3, 1 and 3), and an average pixel value of 3 for the lower right hand spot (the average of 2, 4, 2 and 4).

FIGS. 36a and 36b represent one example of pixel averaging according to the second embodiment of the spot quantification technique, based on original and spot images of cDNA footprints of a mouse lung tissue. Referring to FIG. 36a, each spot contained four pixels and had a spot diameter of 50 microns, and the pitch (spacing between spots) averaged 60 microns. Referring to FIG. 36b, the pixels between the spots were again assigned values of zero. However, the average pixel values for each of the spots included not only the pixels within each spot but the original first layer of pixels surrounding each spot, each having values of 1 as shown in FIG. 36a. This resulted in an average pixel value of 1.5 for the upper left spot (the average of 2, 4, 2, 4, 1. 1,1, 1, 1, 1, 1, 1, 1 and 1), an average pixel value of 1.25 for the upper right spot (the average of 1, 3, 1, 3, 1, 1, 1, 1, 1, 1,1, 1, land 1), an average pixel value of 1.25 for the lower left hand spot (the average of 1, 3, 1, 3, 1, 1, 1, 1, 1, 1,1, 1, 1 and 1), and an average pixel value of 1.5 for the lower right hand spot (the average of 2, 4, 2, 4, 1, 1, 1, 1, 1, 1, 1, 1, 1 and 1).

### Implementation

As explained above, the method of the invention generates a large amount of data for each image on a single tissue optimization slide. Several tissue optimization slides may need to be analyzed in order to optimize permeabilization based on all the pertinent parameters and image properties, and for each of the various cellular tissues. In order to maintain the data in a useful format for current and future use, the optimum values that are generated for each permeabilization parameter and image property, and for each cellular tissue type, are implemented in a way that enables their storage and avoids unnecessary repetition of the method steps. The optimum values for each parameter and each combination of parameters, generated for each image property and each combination of image properties, and for each cellular tissue, may be implemented by storing them in an instrument such as a computer database or, preferably, a computer that has both a database and the capability of further analysis and optimization of the input information.

Accordingly, the present invention includes an instrument for quantitatively optimizing the permeabilization of cellular tissues used for spatial transcriptomics. The instrument includes an apparatus (such as a computer database) for receiving and storing data for at least one parameter that influences permeabilization in a tissue section. Preferably, the instrument includes apparatus for receiving and storing data for two or more parameters, three or more parameters, four or more parameters, or five or more parameters that influence permeabilization in the cellular tissue. The instrument further includes apparatus (such as a computer program) for correlating variations in the at least one parameter with at least one image property that is determinative of permeabilization in the tissue section. The instrument further includes apparatus for determining an optimum m value for the at least one parameter based on the correlation between variations in the parameter and the image property, and apparatus for storing the optimum value of the at least one parameter. Finally, the instrument includes apparatus for informing a user of the optimum value of the at least one parameter.

The apparatus for receiving and storing data can be configured and/or programmed for receiving and storing data for multiple parameters in sequence or simultaneously. The apparatus for informing the user can be configured and/or programmed to inform the user of the optimum values for each of the multiple parameters. Suitably, consistent with the method described herein, the apparatus for receiving and storing data includes apparatus for receiving and storing FFT image data. The apparatus for correlating variations in the at least one parameter with at least one image property can suitably be configured and/or programmed to correlate variations in the at least one parameter with multiple image properties, either in sequence or simultaneously. The multiple image properties can be selected from image brightness, image sharpness, combinations of image brightness and image sharpness, and additional image properties and combinations thereof.

That apparatus for informing a user of the optimum value of the at least one parameter, and/or the optimum values for multiple parameters, can be a computer screen (such as a desktop or laptop screen), a smartphone screen, an audio device with an artificial voice, a voice-activated device, a hand-held tablet, or any combination thereof. As the database inside the instrument gathers and stores increasing amounts of data for different permeabilization conditions and different types of tissue samples, the instrument becomes more and more reliable as a reference source for optimal permeabilization conditions.

Notwithstanding the foregoing description or the appended claims, the disclosure set forth herein is also defined by the following numbered clauses, which may be beneficial alone or in combination, with one or more other causes or embodiments. Each of these individually numbered clauses may be used or combined with any of the preceding or following clauses. Thus, these clauses are intended to provide support for all such combinations and is not necessarily limited to specific combinations explicitly provided below:

1. A quantitative method of optimizing the permeabilization of tissue samples used for spatial transcriptomics, comprising the steps of:
a) selecting at least one parameter that affects permeabilization of the tissue sample;
b) preparing a plurality of sections of the tissue sample for permeabilization;
c) permeabilizing the sections of the tissue sample, wherein the permeabilization parameter is varied for the different tissue sections during permeabilization;
d) generating a cDNA footprint of each of the permeabilized tissue sections;
e) imaging the cDNA footprints;
f) determining a level of permeabilization for each tissue section by quantifying at least one image property that is determinative of the level of permeabilization; and
g) correlating the at least one image property with the variations of the permeabilization parameter to determine a value for the parameter that indicates an optimum level of the permeabilization parameter, thereby optimizing the permeabilization conditions for the tissue sample.
   2. The method of clause 1, wherein preparing a plurality of tissue sections comprises placing each tissue section on a separate area on a slide, wherein the slide comprises a plurality of array areas for placing tissue sections, and each array area comprising a plurality of capture probes immobilized thereon.
   3. The method of clause 2, wherein the plurality of capture probes comprise capture domains and after permeabilization analytes are released from the tissue sections and a plurality of the released analytes hybridize to the capture domains.
   4. The method of any one of clauses 1-3, wherein the cDNA footprint is generated by extending the capture domains of the capture probes using the hybridized analytes as a template to create cDNA molecules of the hybridized analytes.
   5. The method of clause 4, wherein the capture domain is extended enzymatically in the presence of one or more fluorescently labelled nucleotides, dATP, dCTP, dGTP or dTTP.
   6. The method of any one of clauses 1-4, wherein the cDNA footprint is generated by hybridizing a primer that is complementary to the analytes hybridized to the capture domains and extending the primer enzymatically using the analytes as a template to create cDNA molecules of the hybridized analytes.
   7. The method of any one of clauses 3-6, wherein the analyte is DNA or RNA.
   8. The method of clause 7, wherein RNA is mRNA.
   9. The method of any one of clauses 2-8, wherein the capture domains comprise a poly(T) sequence, a poly(T) random sequence, a random nucleic acid sequence, a semi-random nucleic acid sequence or a non-random nucleotide sequence.
   10. The method of any one of clauses 1-9, wherein the at least one parameter selected is permeabilization time, permeabilization temperature, type of permeabilization reagent, a fixing agent used to fix the cellular tissue before permeabilization, and a staining agent used to stain the cellular tissue before permeabilization.
   11. The method of clause 10, wherein the permeabilization time is varied in selected increments between about 5 minutes and about 60 minutes.
   12. The method of clause10, wherein the permeabilization temperature is varied in selected increments between about 4 degrees and about 50 degrees C.
   13. The method of clause 10, wherein the type of permeabilization reagent is varied between one or more of organic solvents, cross-linking agents, detergents, enzymes, lysis reagents, and combinations thereof.
   14. The method of clause 13, wherein the organic solvent is varied between two or more of acetone, methanol, ethanol, and combinations thereof.
   15. The method of clause 13, wherein the crosslinking agent is varied between paraformaldehyde, glutaraldehyde, and combinations thereof.
   16. The method of clause 13, wherein the detergent is varied between two or more of saponin, Triton X-100™ (C₁₄—H₂₂—O—(C₂—H₄—O)ₙ), Tween-20™ (polyoxyethylene sorbitan monolaurate), sodium dodecyl sulfate, N-lauroylsarcisine, N-lauroylsarcisine sodium salt, and combinations thereof.
   17. The method of clause 13, wherein the enzyme is varied between trypsin, proteases, and combinations thereof.
   18. The method of clause 13, wherein the lysis reagent is varied between two or more of lysozymes, achromopeptidase, lysostaphin, labiase, kitalase, lyticase, and combinations thereof.
   19. The method of any one of clauses 13-18, wherein the permeabilization reagent further comprises varying the concentration of the permeabilization reagent.
   20. The method of clause 19, wherein the concentration of permeabilization agent is varied in increments between about 1.0% w/v and about 15% w/v.
   21. The method of clause 10, wherein the fixing agent is varied between two or more of an alcohol, ketone, aldehyde, cross-linking agent, disuccinimidyl suberate (DSS), dimethylsuberimidate (DMS), formalin, dimethyladipimidate (DMA), dithio-bis (-succinimidyl propionate) (DSP), disuccinimidyl tartrate (DST), ethylene glycol bis (succinimidyl succinate) (EGS), and combinations thereof.
   22. The method of clause 10, wherein the staining agent is varied between two or more of acridine orange, Bismarck brown, carmine, Coomassie blue, cresyl violet, 4,6-diamidino-2-phenylindole (DAPI), eosin, hematoxylin, hematoxylin & eosin (H&E), ethidium bromide, acid fuchsine, iodine, methyl green, bisbenzimides, iodine, methyl green, methylene blue, neutral red, Nile blue, Nile red, osmium tetroxide, propidium iodide, rhodamine, safrain, radioisotopes, fluorophores, chemiluminescent compounds, bioluminescent compounds, and combinations thereof.
   23. The method of any one of clauses 1-22, wherein the at least one image property comprises image brightness, image sharpness, or both.
   24. The method of any one of clauses 1-23, wherein the at least one image property is quantified by obtaining at least one image signal for each permeabilized tissue section representing a variation in the at least one parameter, determining an intensity for each image signal, and preparing a plot of the image signal intensities versus the variations in the parameter.
   25. The method of any one of clauses 1-24, wherein the at least one image property is quantified by obtaining a first image of each permeabilized tissue section and the array area on which it is placed, applying a mask to the area surrounding the tissue section on the array area, obtaining a mask image of each tissue section and the surrounding array area, wherein the mask minimizes or eliminates background region signal from the array area surrounding the tissue section, and merging each first image and each corresponding mask image to obtain a tissue image for each of the permeabilized tissue sections.
   26. The method of clause 25, further comprising the steps of preparing a first plot of pixel count versus image intensity for each first image, preparing a second plot of pixel count verses image intensity for each mask image, and preparing a third plot of pixel count versus image intensity for each tissue section.
   27. The method of any one of clauses 1-26, wherein the at least one image property is quantified using Fast Fourier Transform (FFT).
   28. The method of any one of clauses 1-27, further comprising the steps of obtaining an FFT image of each permeabilized tissue section, and processing and comparing the FFT images for each of the permeabilized tissue sections.
   29. The method of clause 28, wherein the FFT images are processed by extracting a radial profile of each image, by obtaining a power spectrum of each image, or both.
   30. The method any one of clauses 28 or 29, further comprising the step of calculating at least one metric to determine the resolution of each FFT image.
   31. The method of clause 30, wherein the at least one metric comprises an average frequency over a selected range of frequencies, an average intensity of a selected range of frequencies, and a ratio of frequencies over a plurality of selected ranges.
   32. The method of any one of clauses 1-31, wherein the tissue sample is from a mammal, from one or more of a mammalian brain, olfactory bulb, spleen, lung, esophagus, skin, liver, reproductive organ, heart, abdomen and intestines.
   33. A quantitative method of optimizing the permeabilization of tissue samples used for spatial transcriptomics, comprising the steps of:
a) selecting two or more parameters that influence permeabilization in the tissue sample;
b) preparing a first plurality of sections of the tissue sample for permeabilization;
c) permeabilizing the first plurality of sections of the tissue sample, wherein the first of the two or more permeabilization parameters is varied for the different tissue sections;
d) generating a cDNA footprint of each of the first plurality of permeabilized tissue sections,
e) imaging the cDNA footprint for each of the tissue sections;
f) determining a level of permeabilization for each tissue section of the first plurality of tissue sections by quantifying at least one image property that is determinative of permeabilization;
g) correlating the at least one image property with the variations in the first parameter to determine a first value for the first parameter that yields an optimum level of permeabilization based on the first parameter;
h) preparing a second plurality of tissue sections of tissue sample for permeabilization;
i) permeabilizing the second plurality of tissue sections, wherein the second of the two permeabilization parameters is varied for the different tissue sections;
j) generating a cDNA footprint of each of the second plurality of permeabilized tissue sections;
k) imaging the cDNA footprint for each of the tissue sections;
l) determining a level of permeabilization for each tissue section of the second plurality of tissue sections by quantifying at least one image property that is determinative of permeabilization; and
m) correlating the at least one image property with the variations in the second parameter to determine a second value for the second parameter that yields an optimum level of permeabilization based on the second parameter, and based on the first and second values determine the optimal permeabilization of the tissue sample.
   34. The method clause 33, further comprising the steps of:
a) preparing a third plurality of tissue sections of the tissue sample for permeabilization;
b) varying a third parameter at different sections of the third plurality during permeabilization;
c) determining a level of permeabilization for each section of the third plurality of sections of the tissue sample by quantifying at least one image property that is determinative of permeabilization;
d) correlating the at least one image property with the variations in the third parameter to determine a third value for the third parameter that yields an optimum level of permeabilization based on the third parameter; and
e) implementing the third value along with the first and second values to determine the optimal permeabilization of the tissue sample.
   35. The method of clause 34, further comprising the steps of:
a) preparing a fourth plurality of sections of the tissue sample for permeabilization;
b) varying a fourth of the two or more parameters at different sections of the fourth plurality during permeabilization;
c) determining a level of permeabilization for each section of the fourth plurality of sections of the tissue sample by quantifying at least one image property that is determinative of permeabilization;
d) correlating the at least one image property with the variations in the fourth parameter to determine a fourth value for the fourth parameter that yields an optimum level of permeabilization based on the fourth parameter; and
e) implementing the fourth value along with the first, second and third values to determine the optimal permeabilization of the tissue sample.
   36. The method of clause 35, comprising varying at least a sixth of the one or more parameters and repeating steps a-e.
   37. The method of any of clauses 33-36, wherein the at least one image property comprises image brightness, image sharpness, or both.
   38. The method of any one of clauses 33-37, wherein the two or more parameters are selected from permeabilization time, permeabilization temperature, permeabilization reagent, tissue fixing agent, tissue staining agent, and combinations thereof.
   39. The method of clause 38, wherein the two or more parameters are varied sequentially or simultaneously.
   40. The method clause 33, wherein the at least one image property is quantified by obtaining at least one image signal for each permeabilized tissue section representing a variation in each of the two or more parameters, determining an intensity for each image signal, and preparing plots of the image intensities versus the variations in each parameter.
   41. The method of clause 33, wherein the at least one image property is quantified by obtaining a first image of each permeabilized tissue section and the array area on which it is placed, applying a mask to the area surrounding the tissue section on the array area, obtaining a mask image of each tissue section and the surrounding array area, wherein the mask minimizes or eliminates background region signal from the array area surrounding the tissue section, and merging the first image and each corresponding mask image to obtain a tissue image for each of the permeabilized tissue sections.
   42. The method of clause 41, further comprising the steps of preparing a first plot of pixel count versus image intensity for each first image, preparing a second plot of pixel count verses image intensity for each mask image, and preparing a third plot of pixel count versus image intensity for each tissue image.
   43. The method of any one of clauses 33-42, wherein the at least one image property is quantified using Fast Fourier Transform (FFT), further comprising the steps of obtaining an FFT image of each permeabilized tissue section and processing and comparing the FFT images for each of the tissue sections.
   44. The method of clause 43, wherein the FFT images are processed by one or more of extracting a radial profile of each image and obtaining a power spectrum of each image.
   45. The method of one of clauses 43 and 44, further comprising the step of calculating at least one metric to determine the resolution of each image, wherein the at least one metric includes an average frequency over a selected range of frequencies, an average intensity of a selected range of frequencies, and a ratio of frequencies over a plurality of selected ranges.
   46. An instrument for quantitatively optimizing the permeabilization of tissue samples used for spatial transcriptomics, comprising:
a) an apparatus for receiving and storing data for at least one parameter that influences permeabilization in the tissue sample;
b) an apparatus for correlating variations in the at least one parameter with at least one image property that is determinative of permeabilization in the tissue sample;
c) an apparatus for determining an optimum value for the at least one parameter based on the correlation between the variations in the parameter and the image property;
d) an apparatus for storing the optimum value of the at least one parameter; and
e) an apparatus for informing a user of the optimum value of the at least one parameter.
   47. The instrument of clause 46, wherein the apparatus for receiving and storing data comprises receiving and storing data for multiple parameters simultaneously, receiving and storying FFT image data, and the apparatus for informing a user comprises informing the user of optimum values for each of the multiple parameters.
   48. The instrument of clause 46, wherein the at least one parameter is selected from the group consisting of permeabilization time, permeabilization temperature, permeabilization reagent, tissue fixing agent, tissue staining agent, and combinations thereof.
   49. The instrument of clause 46, wherein the apparatus for correlating variations in the at least one parameter with at least one image property comprises apparatus for correlating variations in the at least one parameter with multiple image properties that are determinative of permeabilization, wherein the at least one image property is image brightness, image sharpness, or both.
   50. The method of one of clauses 23 and 37, or the instrument of clause 49, wherein the image brightness is quantified by determining a pixel intensity distribution for each variation in the parameter, and wherein the step of correlating the image brightness with the variations in the parameter comprises the step of plotting a graph of pixel intensity distribution versus the variations in the parameter.
   51. The method of one of clauses 23 and 37, or the instrument of clause 49, wherein the image brightness is quantified by converting the photomicrographic images into spot images and analyzing pixel intensities of an array of spots within each spot image.
   52. The method of clause 51, wherein the step of analyzing pixel intensities comprises determining an average pixel intensity for pixels within each spot in the array and assigning a pixel value of zero to pixels present in spaces between the spots, or determining an average of pixel intensities comprises determining an average pixel intensity for pixels within each spot and pixels within a defined vicinity of each spot.
   53. The method of one of clauses 23 and 37, or the instrument of clause 49, wherein the image sharpness is quantified by one or more of
a) determining the absolute central moment for each variation in the parameter,
b) determining the Brenner gradient for each variation in the parameter,
c) determining the image contrast and/or the image contrast A for each variation in the parameter,
d) determining the image curvature for each variation in the parameter,
e) determining the DCT energy ratio, DCT reduced energy ratio, DCT energy ratio A and/or DCT reduced energy ratio A for each variation in the parameter,
f) determining the gaussian derivative for each variation in the parameter,
g) determining the gray level variance and/or the gray level local variance for each variation in the parameter,
h) determining the normalized GLV for each variation in the parameter,
i) determining the energy of gradient for each variation in the parameter,
j) determining the thresholded gradient for each variation in the parameter,
k) determining the squared gradient for each variation in the parameter,
l) determining the Hemli's mean for each variation in the parameter,
m) determining the histogram entropy and/or the histogram range for each variation in the parameter,
n) by determining the energy of laplacian and/or the modified lablacian, and/or the variance of laplacian, and/or the diagonal lapalcian for each variation in the parameter,
o) determining the steerable filters for each variation in the parameter,
p) determining the spatial frequency for each variation in the parameter,
q) determining the Tenengrad value and/or Tenengrad variance for each variation in the parameter,
r) determining the Vollath's correlation for each variation in the parameter,
s) determining the sum of wavelet coefficients, the sum of wavelet coefficients A, and/or the variance of wavelets for each variation in the parameter, and
t) determining the multi-level two-dimensional inverse FWT and/or the multi-level two-dimensional FWT A for each variation in the parameter.
   54. The method of any one of clauses 1-45, wherein the step of correlating the at least one image property with the variations in the parameter comprises plotting a plurality of image properties versus the variations in the parameter using a heatmap.
   55. The method of any one of clauses 23 and 38, or the instrument of clause 49, wherein the image brightness is quantified by determining a pixel intensity distribution for each variation in the parameter.
   56. The method of clause 55, wherein the step of correlating the image brightness with the variations in the parameter comprises the step of plotting a graph of pixel intensity distribution versus the variations in the parameter.
   57. A quantitative method of optimizing the permeabilization of tissue samples used for spatial transcriptomics, comprising the steps of:
a) selecting two or more parameters that influence permeabilization in the tissue sample;
b) preparing a first plurality of sections of the tissue sample for permeabilization;
c) permeabilizing the first plurality of sections of the tissue sample, wherein the first of the two or more permeabilization parameters is varied for the different tissue sections;
d) generating a cDNA footprint of each of the first plurality of permeabilized tissue sections,
e) imaging the cDNA footprint for each of the tissue sections;
f) determining a level of permeabilization for each tissue section of the first plurality of tissue sections by quantifying at least one image property that is determinative of permeabilization;
g) correlating the at least one image property with the variations in the first parameter to determine a first value for the first parameter that yields an optimum level of permeabilization based on the first parameter;
h) performing steps b) through g) with a second plurality of sections of the tissue sample; and
i) correlating the at least one image property with the variations in the second parameter to determine a second value for the second parameter that yields an optimum level of permeabilization based on the second parameter, and based on the first and second values determine the optimal permeabilization of the tissue sample.
   58. The method of clause 57, further comprising the steps of:
a) preparing a third plurality of tissue sections of the tissue sample for permeabilization;
b) varying a third parameter at different sections of the third plurality during permeabilization;
c) determining a level of permeabilization for each section of the third plurality of sections of the tissue sample by quantifying at least one image property that is determinative of permeabilization;
d) correlating the at least one image property with the variations in the third parameter to determine a third value for the third parameter that yields an optimum level of permeabilization based on the third parameter; and
e) implementing the third value along with the first and second values to determine the optimal permeabilization of the tissue sample.
   59. The method of clause 58, further comprising the steps of:
a) preparing a fourth plurality of sections of the tissue sample for permeabilization;
b) varying a fourth of the two or more parameters at different sections of the fourth plurality during permeabilization;
c) determining a level of permeabilization for each section of the fourth plurality of sections of the tissue sample by quantifying at least one image property that is determinative of permeabilization;
d) correlating the at least one image property with the variations in the fourth parameter to determine a fourth value for the fourth parameter that yields an optimum level of permeabilization based on the fourth parameter; and
e) implementing the fourth value along with the first, second and third values to determine the optimal permeabilization of the tissue sample.
   60. The method of clause 59, comprising varying at least a sixth of the one or more parameters and repeating steps a-e.
   61. The method of clause 57, wherein the at least one image property comprises image brightness, image sharpness, or both.
   62. The method of clause 61, wherein the image sharpness is quantified by one or more of
a) determining the absolute central moment for each variation in the parameter,
b) determining the Brenner gradient for each variation in the parameter,
c) determining the image contrast and/or the image contrast A for each variation in the parameter,
d) determining the image curvature for each variation in the parameter,
e) determining the DCT energy ratio, DCT reduced energy ratio, DCT energy ratio A and/or DCT reduced energy ratio A for each variation in the parameter,
f) determining the gaussian derivative for each variation in the parameter,
g) determining the gray level variance and/or the gray level local variance for each variation in the parameter,
h) determining the normalized GLV for each variation in the parameter,
i) determining the energy of gradient for each variation in the parameter,
j) determining the thresholded gradient for each variation in the parameter,
k) determining the squared gradient for each variation in the parameter,
l) determining the Hemli's mean for each variation in the parameter,
m) determining the histogram entropy and/or the histogram range for each variation in the parameter,
n) by determining the energy of laplacian and/or the modified laplacian, and/or the variance of laplacian, and/or the diagonal laplacian for each variation in the parameter,
o) determining the steerable filters for each variation in the parameter,
p) determining the spatial frequency for each variation in the parameter,
q) determining the Tenengrad value and/or Tenengrad variance for each variation in the parameter,
r) determining the Vollath's correlation for each variation in the parameter,
s) determining the sum of wavelet coefficients, the sum of wavelet coefficients A, and/or the variance of wavelets for each variation in the parameter, and
t) determining the multi-level two-dimensional inverse FWT and/or the multi-level two-dimensional FWT A for each variation in the parameter.
   63. The method of clause 61, wherein the image brightness is quantified by:
a) determining a pixel intensity distribution for each variation in the parameter, and wherein the step of correlating the image brightness with the variations in the parameter comprises the step of plotting a graph of pixel intensity distribution versus the variations in the parameter; or
b) converting the photomicrographic images into spot images and analyzing pixel intensities of an array of spots within each spot image.
   64. The method of clause 63, wherein the step of analyzing pixel intensities comprises determining an average pixel intensity for pixels within each spot in the array and assigning a pixel value of zero to pixels present in spaces between the spots, or determining an average of pixel intensities comprises determining an average pixel intensity for pixels within each spot and pixels within a defined vicinity of each spot.
   65. The method of clause 57, wherein the two or more parameters are:
a) selected from permeabilization time, permeabilization temperature, permeabilization reagent, tissue fixing agent, tissue staining agent, and combinations thereof; or
b) varied sequentially or simultaneously.
   66. The method of clause 65, wherein the image brightness is quantified by determining a pixel intensity distribution for each variation in the parameter.
   67. The method of clause 66, wherein the step of correlating the image brightness with the variations in the parameter comprises the step of plotting a graph of pixel intensity distribution versus the variations in the parameter.
   68. The method of clause 57, wherein the step of correlating the at least one image property with the variations in the parameter comprises plotting a plurality of image properties versus the variations in the parameter using a heatmap.
   69. The method of clause 57, wherein the at least one image property is:
a) quantified by obtaining at least one image signal for each permeabilized tissue section representing a variation in each of the two or more parameters, determining an intensity for each image signal, and preparing plots of the image intensities versus the variations in each parameter; or
b) quantified by obtaining a first image of each permeabilized tissue section and the array area on which it is placed, applying a mask to the area surrounding the tissue section on the array area, obtaining a mask image of each tissue section and the surrounding array area, wherein the mask minimizes or eliminates background region signal from the array area surrounding the tissue section, and merging the first image and each corresponding mask image to obtain a tissue image for each of the permeabilized tissue sections; or
c) quantified using Fast Fourier Transform (FFT), further comprising the steps of obtaining an FFT image of each permeabilized tissue section and processing and comparing the FFT images for each of the tissue sections.
   70. The method of clause 69, further comprising the steps of preparing a first plot of pixel count versus image intensity for each first image, preparing a second plot of pixel count verses image intensity for each mask image, and preparing a third plot of pixel count versus image intensity for each tissue image.
   71. The method of clause 69, wherein the FFT images are processed by one or more of extracting a radial profile of each image and obtaining a power spectrum of each image.
   72. The method of clause 69, further comprising the step of calculating at least one metric to determine the resolution of each image, wherein the at least one metric includes an average frequency over a selected range of frequencies, an average intensity of a selected range of frequencies, and a ratio of frequencies over a plurality of selected ranges.
   73. An instrument for quantitatively optimizing the permeabilization of tissue samples used for spatial transcriptomics, comprising:
a) an apparatus for receiving and storing data for at least one parameter that influences permeabilization in the tissue sample;
b) an apparatus for correlating variations in the at least one parameter with at least one image property that is determinative of permeabilization in the tissue sample;
c) an apparatus for determining an optimum value for the at least one parameter based on the correlation between the variations in the parameter and the image property;
d) an apparatus for storing the optimum value of the at least one parameter; and
e) an apparatus for informing a user of the optimum value of the at least one parameter.
   74. The instrument of clause 73, wherein the apparatus for receiving and storing data comprises receiving and storing data for multiple parameters simultaneously, receiving and storying FFT image data, and the apparatus for informing a user comprises informing the user of optimum values for each of the multiple parameters.
   75. The instrument of clause 73, wherein the at least one parameter is selected from the group consisting of permeabilization time, permeabilization temperature, permeabilization reagent, tissue fixing agent, tissue staining agent, and combinations thereof.
   76. The instrument of clause 73, wherein the apparatus for correlating variations in the at least one parameter with at least one image property comprises apparatus for correlating variations in the at least one parameter with multiple image properties that are determinative of permeabilization, wherein the at least one image property is image brightness, image sharpness, or both.

The embodiments of the invention described herein are exemplary, and various modifications and improvements can be made without departing from the spirit and scope of the invention. The scope of the invention is defined by the appended claims, and all changes that fall within the meaning and range of equivalents are intended to be embraced therein.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding (i) either or (ii) both of those included limits are also included in the invention. For example, “1 to 50,” includes “2 to 25,” “5 to 20,” “25 to 50,” “1 to 10,” etc.

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

What is claimed is:

1. A quantitative method of optimizing the permeabilization of tissue samples used for spatial transcriptomics, comprising the steps of:
a) selecting two or more parameters that influence permeabilization in a tissue sample;
b) preparing a first plurality of sections of the tissue sample for permeabilization;
c) permeabilizing the first plurality of sections of the tissue sample, wherein a first of the two or more permeabilization parameters is varied for the different tissue sections;
d) generating a cDNA footprint of each of the first plurality of permeabilized tissue sections,
e) imaging the cDNA footprint for each of the tissue sections to generate photomicrographic images of the cDNA footprint;
f) determining a level of permeabilization for each tissue section of the first plurality of tissue sections by quantifying at least one image property that is determinative of permeabilization;
g) correlating the at least one image property with the variations in the first parameter to determine a first value for the first parameter that yields an optimum level of permeabilization based on the first parameter;
h) performing steps b) through f) with a second plurality of sections of the tissue sample; and
i) correlating the at least one image property with the variations in the second parameter to determine a second value for the second parameter that yields an optimum level of permeabilization based on the second parameter, and based on the first and second values, determine an optimal permeabilization condition of the tissue sample.
